(19) United States
(12) Patent Application Publication
(10) Pub. No.: US 2010/0028955 A1
(43) Pub. Date: Feb. 4, 2010

(54) Sequence Amplification with Target Primers

(75) Inventors: Kai Qin Lao, Pleasanton, CA (US); Neil Straus, Emeryville, CA (US)

Correspondence Address: LIFE TECHNOLOGIES CORPORATION;C/O INTELLEVATE, P.O. BOX 52050, MINNEAPOLIS, MN, 55402, US

(73) Assignee: LIFE TECHNOLOGIES CORPORATION, Carlsbad, CA (US)

(21) Appl. No.: 12/507,743

(22) Filed: Jul. 22, 2009

Related U.S. Application Data
(60) Provisional application No. 61/082,803, filed on Jul. 22, 2008.

(51) Int. Cl. C12P 19/34; C12N 9/00

(52) U.S. Cl. 435/91.2; 435/183

(57) ABSTRACT

The present disclosure relates to the amplification of target nucleic acid sequences for various sequencing and/or identification techniques. This can be accomplished via the use of target primers and isothermal multiple strand displacement (MDA) processes. The use of these target primers and MDA, as described herein, allows for, for example, the reduction in the amplification of undesired hybridization events (such as primer dimerization and the “jackpot mutation” effect of PCR) while allowing for the amplification of the target nucleic acid sequences.

### CROSS-REFERENCE TO RELATED APPLICATIONS

This application claims a priority benefit under 35 U.S.C. §119(e) to U.S. Patent Application No. 61/082,803, filed Jul. 22, 2008, which is incorporated herein by reference in its entirety.

### FIELD OF INVENTION

The invention relates to methods and compositions for amplifying nucleic acid sequences.

### INTRODUCTION

Whole genome amplification (WGA) can be a valuable technique for amplification of a genome from minimal or limiting amounts of DNA for subsequent molecular genetic analysis.

Whole genome amplification can be performed using either conventional or nonconventional PCR amplification methods. Conventional PCR entails the amplification and subsequent detection of specific DNA sequences which are precisely characterized in length and sequence using nondegenerate primers, while random, “non-conventional” PCR involves universal amplification of prevailing DNA or amplification of unknown intervening sequences which are not generally defined in length or sequence using degenerate primers.

### SUMMARY

In some embodiments, a method is provided for genome wide nucleic acid sequence amplification. The method can comprise, consist, or consist essentially of providing a first primer that comprises a 3′ target specific region and a universal region and contacting the first primer and a target nucleic acid sequence such that the 3′ target specific region hybridizes to the target nucleic acid sequence. The method further comprises, consists, or consists essentially of performing an isothermal multiple strand displacement amplification on the target nucleic acid sequence using the first primer, and forming a double-extended primer comprising the universal region on one end and a sequence that is complementary to the universal region on an opposite end. In some embodiments, the method further comprises adding at least a third primer that is complementary to a sequence within the insert section, and performing PCR amplification within the insert section.

In some embodiments, a method is provided for genome wide nucleic acid sequence amplification. The method can comprise, consist, or consist essentially of providing a target primer that comprises a 3′ target specific region and a universal region. The 3′ target specific region can comprise a degenerate sequence. The method can further comprise, consist, or consist essentially of contacting the target primer to a target nucleic acid sequence such that the 3′ target specific region hybridizes to the target nucleic acid sequence and performing an isothermal multiple strand displacement amplification on the target nucleic acid sequence using the target primer. Following the isothermal multiple strand displacement amplification, but prior to the formation of a significant amount of a hyper-branched product, in the isothermal multiple strand displacement amplification one can stop the isothermal multiple strand displacement and perform a PCR amplification, thereby forming a double-extended target primer comprising the universal region on one end and a sequence that is complementary to the universal region on an opposite end. The method can further comprise, consist, or consist essentially of performing an amplification of the double extended target primer using an amplification primer. The amplification primer can comprise, consist, or consists essentially of a universal region. The method can further comprise, consist, or consist essentially of adding at least a first and second insert primer, and performing PCR amplification within the insert section, using the first and second insert primers.

In some embodiments, the target primer includes a random or degenerate region. In some embodiments, the target primer includes a noncomplementary region to reduce the likelihood of nonspecific hybridization of the primer (such as primer dimers) during subsequent amplification is provided.

In some embodiments, a PCR primer kit or a primer is provided. The kit or target primer can comprise a universal region. The universal region can comprise a nucleic acid sequence that has an appropriate Tm and GC content to serve as a primer. The universal region comprises 12 to 35 bases. The target primer further comprises a 3′ target specific region located in the 3′ direction from the universal region, wherein the 3′ target specific region comprises at least 2 bonds that are phosphothioate bonds.

### BRIEF DESCRIPTION OF THE DRAWINGS

FIG. 1A depicts one embodiment of a linear target primer.

FIG. 1B is a flow chart depicting one embodiment using a linear target primer to produce a self-hybridizing nucleic acid sequence.

FIG. 1C depicts one embodiment of a loopable target primer.

FIG. 1D is a flow chart depicting one embodiment using a loopable target primer to produce a self-hybridizing nucleic acid sequence.

FIG. 1E is a flow chart depicting some embodiments involving a target primer.

FIG. 2 depicts an embodiment of using a target primer.

FIG. 3 depicts an embodiment of using a target primer.

FIG. 4 depicts an embodiment of using a target primer.

FIG. 5 depicts an embodiment of using a target primer.

FIG. 6 depicts an embodiment of using a target primer.

FIG. 7 depicts an embodiment of using a target primer.

FIG. 8 depicts an embodiment of a MDA and/or PCR technique.

FIG. 9 depicts a flow chart of various embodiments of MDA and/or PCR target primer amplification.

### DETAILED DESCRIPTION OF VARIOUS EMBODIMENTS

The use of various primers in the amplification of a target nucleic acid sequence is described herein. In some embodiments, this involves the use of primers to put complementary sequences on both ends of target nucleic acid sequences. Products that include insert sections that are very short (including no insert sections, such as primer dimers) will self-terminate from subsequent amplification, as they will rapidly self-hybridize. Products that include an insert section that is relatively long can remain viable templates for continued amplification. Thus, primer dimers and other short products are selectively removed from the amplification process.

In some embodiments, this ability to selectively amplify longer sequences is employed within a hybrid isothermal multiple strand displacement (“MDA”)/PCR amplification reaction. In some embodiments, this allows one to obtain the benefits of a MDA reaction and a PCR reaction, without the downside of primer dimers or other short products that could otherwise overrun the reactions. Thus, in some embodiments, the invention includes a process that can reduce the primer induced background present in MDA while retaining relatively even gene amplification of MDA.

In some embodiments, the above process generates genome wide amplification of DNA that can be readily amplified with routine forms of PCR. In some embodiments, this process avoids the “jackpot mutation” effect of PCR on single copy molecules because the PCR can be performed at a multicopy level. A “jackpot mutation” effect of PCR refers generally to the generation of PCR products by amplification of a single molecule of template as opposed to PCR amplification from multiple molecules of template. In some embodiments, one or more of the above advantages, can be provided by at least one of the presently disclosed embodiments.

In some embodiments, the above approach is applied to genomic analysis with especially advantageous results. For example, previously, to get around various background issues involving primer dimmers in MDA amplification methods, one would used constrained random primers of randomized A, G sequences, where thymine and/or cytosine would be excluded from the priming region. Even the addition of one such base (e.g., T), to the constrained random priming significantly degrades the resulting product. Another approach that had been used in MDA was to constrain the reaction to very small volumes, 60 nl or less, using microfluidic devices. As some of the above techniques avoid these issues some of the presently disclosed embodiments address some or all of the above problems without some or all of the previous constraints. Thus, in some embodiments, random or degenerate priming regions can include nucleotides other than A and/or G (and simply not be constrained). Furthermore, in some embodiments, larger volumes (e.g., above 60 nl) can be used during amplification.

The above and additional embodiments are described in greater detail below. Following the definition and alternative embodiments section provided immediately below, a general description of how target primers generally work is provided. Following this section, especially advantageous embodiments involving a hybrid MDA/PCR amplification process are described. Following these sections, a brief description providing additional embodiments is provided along with a series of specific examples.

### Some Definitions and Alternative Embodiments:

As used herein, the term “target nucleic acid sequence” refers to a polynucleotide sequence that is sought to be detected, sequenced, and/or characterized in a sample. The target nucleic acid sequence can be obtained from any source and can include any number of different compositional components. For example, the target can be nucleic acid (e.g. DNA or RNA), transfer RNA, siRNA, and can include nucleic acid analogs or other nucleic acid mimic. The target can be methylated, non-methylated, or both. The target can be bisulfite-treated and can contain non-methylated cytosines converted to uracil. Further, it will be appreciated that “target nucleic acid sequence” can refer to the target nucleic acid sequence itself, as well as surrogates thereof, for example amplification products, and native sequences. In some embodiments, the target nucleic acid sequence is a miRNA molecule. In some embodiments, the target nucleic acid sequence lacks a poly-A tail. In some embodiments, the target nucleic acid sequence is a short DNA molecule derived from a degraded source, such as can be found in, for example but not limited to, forensics samples (see for example Butler, 2001, Forensic DNA Typing: Biology and Technology Behind STR Markers). In some embodiments, the target nucleic acid sequences of the present teachings can be present or derived from any of a number of sources, including without limitation, viruses, prokaryotes, eukaryotes, for example but not limited to plants, fungi, and animals. These sources can include, but are not limited to, whole blood, a tissue biopsy, lymph, bone marrow, amniotic fluid, hair, skin, semen, biowarfare agents, anal secretions, vaginal secretions, perspiration, saliva, buccal swabs, various environmental samples (for example, agricultural, water, and soil samples), research samples generally, purified samples generally, cultured cells, and lysed cells.

It will be appreciated that target nucleic acid sequences can be isolated or obtained from samples using any of a variety of procedures known in the art, for example the Applied Biosystems ABI Prism™ 6100 Nucleic Acid PrepStation, and the ABI Prism™ 6700 Automated Nucleic Acid Workstation, Boom et al., U.S. Pat. No. 5,234,809, mirVana RNA isolation kit (Ambion), etc. It will be appreciated that target nucleic acid sequences can be cut or sheared prior to analysis, including the use of such procedures as mechanical force, sonication, heat, restriction endonuclease cleavage, or any method known in the art. Cleaving can be done specifically or non-specifically. In general, the target nucleic acid sequences of the present teachings will be single stranded, though in some embodiments the target nucleic acid sequence can be double stranded, and a single strand can be produced by denaturation. In some embodiments, the target nucleic acid sequence is genomic DNA.

As will be appreciated by one of skill in the art, the term “target nucleic acid sequence” can have different meanings at different points throughout the method. For example, in an initial sample, there can be a target nucleic acid sequence that is 2 kb in length. When this is amplified by the target primer to form a double-extended target primer, part of the target nucleic acid sequence can be contained within the double-extended target primer; however, not all of the target nucleic acid sequence need be contained within the double-extended target primer. Regardless of this, the section of the target nucleic acid sequence that is amplified can still be referred to as the “target nucleic acid sequence” (in part because it will still indicate the presence or absence of the large target nucleic acid sequence of which it is a part). Similarly, when the section of the insert section, which contains the target nucleic acid sequence, is amplified by the insert amplification primers it can also be described as amplifying the “target nucleic acid sequence.” One of skill in the art will appreciate that, likely, the length of the target nucleic acid sequence will decrease as the sequence is processed further. When desired, each target nucleic acid sequence in each step can be specifically designated as an “initial target nucleic acid sequence,” a “double-extended target primer target nucleic acid sequence”, and an “insert section target nucleic acid sequence.” Additionally, one of skill in the art will appreciate that the sequence that one is interested in determining if present in a sample can be a separate sequence from a target nucleic acid sequence that is amplified. For example, the sequences can be in linkage disequilibrium or from a different part of a gene or stretch of nucleic acids. Such sequences can be termed “inquiry target nucleic acid sequences.”

The term “whole genome amplification” does not require that 100% of a genome be amplified. Rather, partial amounts of the genome can be amplified and still qualify as a whole genome amplification process. Thus, the above term simply denotes that amplification across a genome has occurred, and can be interpreted to mean genome-wide amplification. The amplification process is one that amplifies a significant portion of the genomic nucleic acid in a sample. In some embodiments, the significant portion is at least 30%, for example, 30, 30-40, 40-50, 50-60, 60-70, 70-80, 80-90, 90-95, 95-98, 98-99, 99-100% of the genomic nucleic acid in a sample. As will be appreciated by one of skill in the art, the genomic nucleic acid need not be directly derived from a biological host and can itself be the result of some previous manipulation or amplification.

Unless explicitly denoted, the term “target primer” can refer to both or either a “linear primer” or a “loopable primer.” Thus, “target primer” is a genus that includes both “linear primer” and “loopable primer” or “looped primer.”

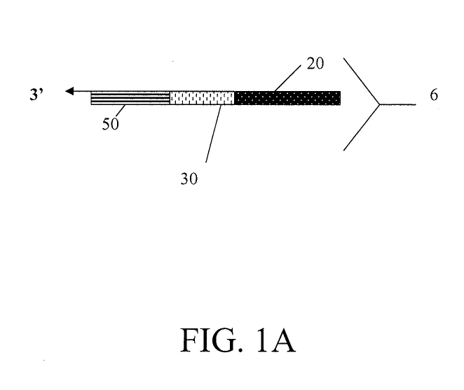

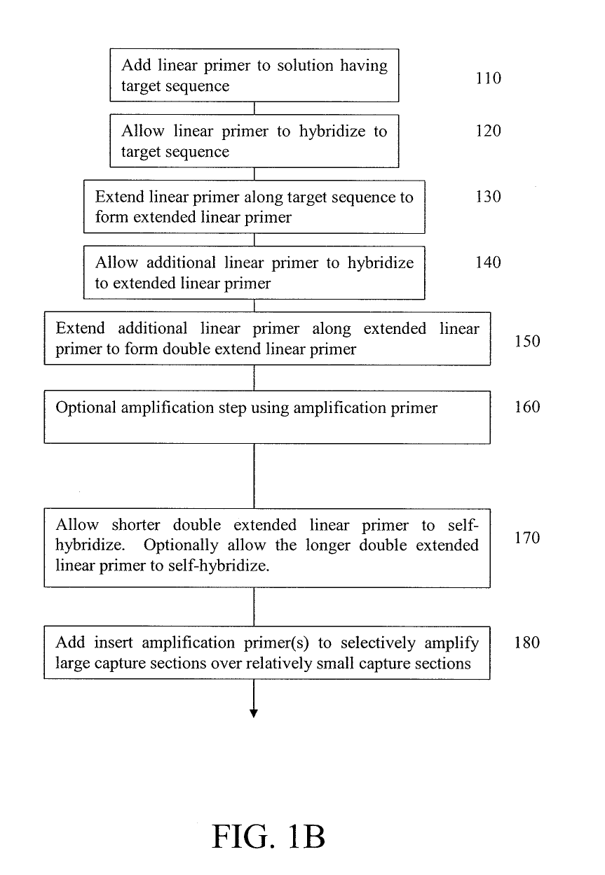

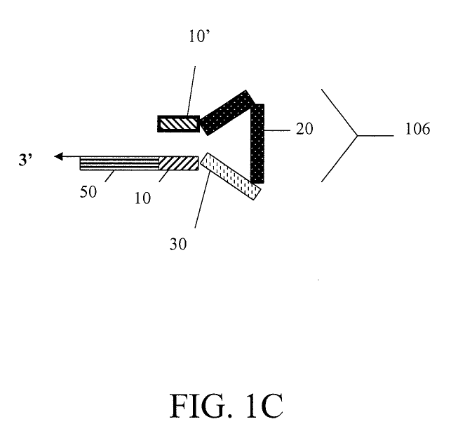

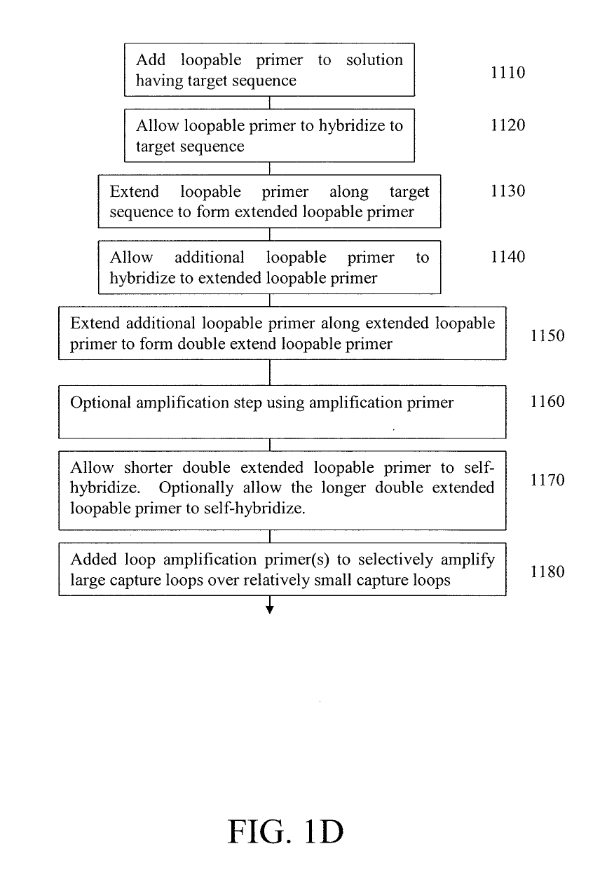

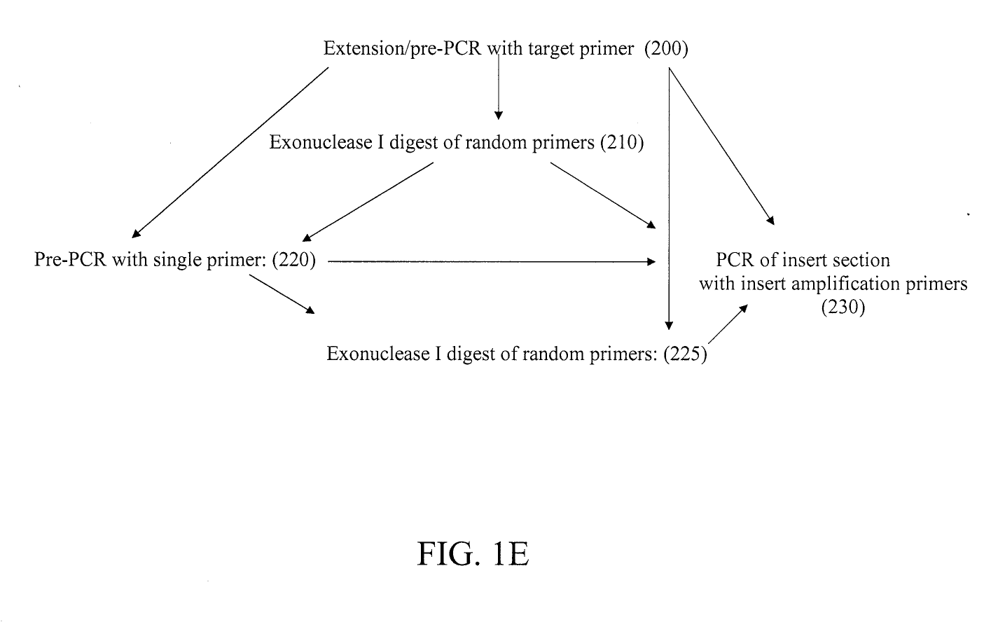

As used herein, the term “loopable primer” or “looped primer” refers to a molecule comprising a 3′ target specific portion, a stem (comprising a first loop forming region and a second loop forming region), and an insert portion (which can optionally include a noncomplementary region and will include a universal region). Illustrative loopable primers are depicted in FIG. 1C and elsewhere in the present teachings. It will be appreciated that the loopable primers can be comprised of ribonucleotides, deoxynucleotides, modified ribonucleotides, modified deoxyribonucleotides, modified phosphate-sugar-backbone oligonucleotides, nucleotide analogs, or combinations thereof. For some illustrative teachings of various nucleotide analogs etc, see Fasman, 1989, Practical Handbook of Biochemistry and Molecular Biology, pp. 385-394, CRC Press, Boca Raton, Fla., Loakes, N. A. R. 2001, vol 29:2437-2447, and Pellestor et al., Int J Mol. Med. 2004 April; 13(4):521-5), references cited therein, and recent articles citing these reviews. It will be appreciated that the selection of the loopable primers to query a given target nucleic acid sequence, and the selection of which collection of target nucleic acid sequences to query in a given reaction with which collection of loopable primers, will involve procedures generally known in the art, and can involve the use of algorithms to select for those sequences with minimal secondary and tertiary structure, those targets with minimal sequence redundancy with other regions of the genome, those target regions with desirable thermodynamic characteristics, and other parameters desirable for the context at hand. In some embodiments, the loop includes one or more additional nucleic acids that serve a desired function. In some embodiments, a universal region is included within the loop. In some embodiments, a noncomplementary region or sequence is included within the loop. In some embodiments, an identifying portion is included within the loop.

As will be appreciated by one of skill in the art, even though a primer is “loopable” it may not always be in its looped form. For example, at high temperatures or salt conditions, the two loop forming and/or complementary regions can separate from one another. However, even in situations where the loopable primer is not actually looped, it can still be referred to as a “loopable primer.” Thus, the term “loopable primer” does not require that the primer actually be in the looped configuration.

As used herein, the term “linear primer” refers to a molecule comprising a 3′ target specific portion and a universal region. It will be appreciated that the linear primers can be comprised of ribonucleotides, deoxynucleotides, modified ribonucleotides, modified deoxyribonucleotides, modified phosphate-sugar-backbone oligonucleotides, nucleotide analogs, or combinations thereof. For some illustrative teachings of various nucleotide analogs etc, see Fasman, 1989, Practical Handbook of Biochemistry and Molecular Biology, pp. 385-394, CRC Press, Boca Raton, Fla., Loakes, N. A. R. 2001, vol 29:2437-2447, and Pellestor et al., Int J Mol. Med. 2004 April; 13(4):521-5), references cited therein, and recent articles citing these reviews. It will be appreciated that the selection of the linear primers to query a given target nucleic acid sequence, and the selection of which collection of target nucleic acid sequence to query in a given reaction with which collection of linear primers, will involve procedures generally known in the art, and can involve the use of algorithms to select for those sequences with desirable features, such as, minimal secondary and tertiary structure, those targets with minimal sequence redundancy with other regions of the genome, those target regions with desirable thermodynamic characteristics, and other parameters desirable for the context at hand. In some embodiments, a universal primer is included within the linear primer. In some embodiments, a noncomplementary region or sequence is included within the linear primer. In some embodiments, an identifying portion is included within the linear primer.

As used herein, the term “3′ target-specific portion” refers to a single stranded portion of a target primer that is complementary to at least a portion of a target nucleic acid sequence. The 3′ target-specific portion is located downstream from the universal region and/or noncomplementary region of the target primer. In some embodiments, the 3′ target-specific portion is between 4 and 15 nucleotides long. In some embodiments, the 3′ target-specific portion is between 6 and 12 nucleotides long. In some embodiments, the 3′ target-specific portion is 7 nucleotides long. It will be appreciated that, in light of the present disclosure, routine experimentation can be used to optimize length, and that 3′ target-specific portions that are longer than 15 nucleotides or shorter than 4 nucleotides are also contemplated by the present teachings. In some embodiments, modified bases such as locked nucleic acids (LNA) can be used in the 3′ target specific portion to increase the stability, for example by increasing the Tm of the target primer (see for example Petersen et al., Trends in Biochemistry (2003), 21:2:74-81). In some embodiments, universal bases can be used in the 3′ target specific portion, for example to allow for smaller libraries of target primers. Universal bases can also be used in the 3′ target specific portion to allow for the detection of unknown targets (e.g., targets for which specific binding sequences are not known). For some descriptions of universal bases, see for example Loakes et al., Nucleic Acids Research, 2001, Volume 29, No. 12, 2437-2447. In some embodiments, modifications including but not limited to LNAs and universal bases can improve reverse transcription specificity and potentially enhance detection specificity.

In some embodiments, the 3′ target-specific region includes or is a degenerate region, a random region, a specific region, or a known sequence. In some embodiments, the 3′ target specific region includes a combination of these regions. In some embodiments, the 3′ target specific regions have a Tm of between about 5° C. and 50° C. In some embodiments, a 15-mer has a Tm of less than about 60° C.

The term “degenerate primer” when used herein refers to a mixture of similar primers with differing bases at the varying positions (Mitsuhashi M, J Clin Lab Anal, 10(5):285 93 (1996); von Eggeling et al., Cell Mol Biol, 41(5):653 70 (1995); (Zhang et al., Proc. Natl. Acad. Sci. USA, 89:5847 5851 (1992); Telenius et al., Genomics, 13(3):718 25 (1992)). Such primers can include inosine, as inosine is able to base pair with adenosine, cytosine, guanine or thymidine. Degenerate primers allow annealing to and amplification of a variety of target sequences that can be related. Degenerate primers that anneal to target DNA can function as a priming site for further amplification. A degenerate region is a region of a primer that varies, while the rest of the primer can remain the same. Degenerate primers (or regions) denote more than one primer and can be random. A random primer (or regions) denotes that the sequence is not selected, and it can be degenerate but does not have to be. In some embodiments, the 3′ target specific regions have a Tm of between about 5° C. and 50° C. In some embodiments, a 15-mer has a Tm of less than about 60° C.

A “specific region” (in contrast to a “3′ target specific region” which is a broader genus) is able to bind to a genomic sequence occurring in a genome at a particular frequency. In some embodiments, this frequency is between about 0.01% and 2.0%, such as, between about 0.05% and 0.1% or between about 0.1% and 0.5%. In some embodiments, the length of the “specific region” of a primer depends mainly on the averaged lengths of the predicted PCR products based on bioinformatic calculations. The definition includes, without limitation, a “specific region” of between about 4 and 12 bases in length. In more particular embodiments, the length of the 3′ specific region can be, for example, between about 4 and 20 bases, or between about 8 and 15 bases. Specific regions having a Tm of between about 10° C. and 60° C. are included within the definition. The term, “specific primer,” when used herein refers to a primer of specified sequence.

The term “random region” as used herein refers to a region of an oligonucleotide primer that is able to anneal to unspecified sites in a group of target sequences, such as in a genome. The “random region” facilitates binding of the primer to target DNA and binding of the polymerase enzyme used in PCR amplification to the duplex formed between the primer and target DNA. The random region nucleotides can be degenerate or non-specific, promiscuous nucleobases or nucleobase analogs. The length of the “random region” of the oligonucleotide primer, among other things, depends on the length of the specific region. In certain embodiments, without limitation, the “random region” is between about 2 and 15 bases in length, between about 4 and 12 bases in length or between about 4 and 6 bases in length. In another embodiment, the specific and random regions combined will be about 9 bases in length, e.g., if the specific region has 4 bases, the random region will have 5 bases.

In some embodiments, the 3′ target-specific portion comprises both a specific region and a random region or degenerate region. In other embodiments, the 3′ target-specific portion includes a specific region, and a random region or a degenerate region. In other embodiments, the 3′ target specific region of the target primer only includes a specific region, a random region, or a degenerate region. Of course, known regions (sequences that are known) can also be used or part of the options disclosed herein.

In some embodiments, the term “universal region,” “universal primer region,” or “universal priming region” as used herein refers to a region of an oligonucleotide primer that is designed to have no significant homology to any segment in the genome. However, given that a noncomplementary region can be included in the target primer, nonspecific priming can be further reduced; thus, a universal region is not necessarily required for all embodiments. In some embodiments, the universal region is a region that allows for priming with a known primer. In some embodiments, this primer is common to at least one other nucleic acid sequence. In some embodiments, the “universal region” meets all the requirements for a normal oligonucleotide primer, such as lack of secondary structure, an appropriate Tm, and an appropriate GC content and can be between about 12 and 35 bases in length, between about 15 and 25 bases in length or between about 18 and 22 bases in length. However, as will be appreciated by one of skill in the art, the universal region, when part of the target primer, will be part of a larger structure. Additionally, because the universal region will be part of a larger primer, the universal region need only function as part of the entire target primer. As such, in these embodiments, the universal region need only assist in priming, as described in detail below. In some embodiments, the universal region functions independently as a priming site. In some embodiments, the universal region is the same as the noncomplementary region or they share some of the same nucleic acid sequences. “Universal priming site” when used herein refers to a “universal region” of a primer that can function as a site to which universal primers anneal for priming of further cycles of DNA amplification. In some embodiments, the target primer includes a universal region. The term “universal primer” as used herein refers to a primer that consists essentially of a “universal region”. However, in some embodiments larger primers can comprise a universal region.

As used herein, the “noncomplementary region” refers to a nucleic acid sequence in a target primer or product thereof. In some embodiments, the noncomplementary region is a sequence that is present in at least some of the various primers or sequences in a reaction mixture. In some embodiments, the sequence is common in all or less than all of the primers used, for example 100, 100-99, 99-95, 95-90, 90-80, 80-70, 70-60, 60-50, 50-40, 40-30, 30-20, 20-10, 10-5, 5-1, 1% or less. Thus, in some embodiments, the primers for the target amplification all contain the same noncomplementary sequence. In some embodiments, the primers in subsequent steps (or a percent as noted above) also have the same noncomplementary region. As will be appreciated by one of skill in the art, the presence of similar sequences across various primers will reduce the likelihood that primer dimerization will occur (as the primers will be less likely to hybridize to one another). In some embodiments, the noncomplementary region is noncomplementary with respect to sequences in the target nucleic acid sequence. This embodiment is described in more detail below. In some embodiments, the noncomplementary region is both present in various primers (thereby reducing primer dimerization) and noncomplementary to sequences in the target sequences (e.g., a relatively long series of thymines).

The presence of the noncomplementary sequence need not absolutely prevent the occurrence of primer dimerization or other forms of nonspecific hybridization in every situation. In some embodiments, the presence of the noncomplementary region reduces the likelihood of these undesired forms of hybridization from occurring. In some embodiments, any decrease is sufficient, for example, less than 100% of the dimers that would have occurred without the noncomplementary region, e.g., 100-99, 99-98, 98-95, 95-90, 90-80, 80-70, 70-60, 60-50, 50-40, 40-30, 30-20, 20-10, 10-5, 5-1, or less of the original primer dimers will occur when the noncomplementary region is present in the target primer. In some embodiments, the presence of the noncomplementary region decreases likelihood of nonspecific amplification or amplification of undesirably small sections of target nucleic acid sequence. Additionally, while the noncomplementary sequences can be the same in all of the primers or target primers used, they need not be the same. For example, in some embodiments, the noncomplementary regions in different primers, are not the same sequences (e.g., TTTT vs. CCCC). In other embodiments, the noncomplementary regions are similar, but not identical, (e.g., TTTT vs. TTTC). In other embodiments, the noncomplementary regions comprise completely different nucleic acids and/or sequences of nucleic acids; however, they will still reduce the likelihood of various forms of nonspecific hybridization. As will be appreciated by one of skill in the art, the length of the noncomplementary region can vary and the length required can depend on the various reaction conditions and the sequences present in the target sample, variables that can readily be determined and accommodated for by one of skill in the art.

In some embodiments, the noncomplementary region is effective at reducing the nonspecific hybridization of an amplification primer. The amplification primer can have a region that hybridizes to the noncomplementary region (as well as a region that can hybridize to the universal region). Thus, the amplification primer can be more specific for the double-extended target primer products rather than other nonspecific priming events that could occur if the amplification primer only contained a universal region. Thus, in some embodiments, the presence of a noncomplementary region in the target primer can assist in reducing subsequent nonspecific amplification.

In some embodiments, the noncomplementary region is at least 7-15 nucleotides in length. In some embodiments, the noncomplementary region comprises a series of thymine nucleotides. In some embodiments, the noncomplementary region is 8-12 thymines. In some embodiments, the noncomplementary region includes 5, 6, 7, 8, 9, 10, 11, 12, 13, 14, 15, 16, 17, 18, 19, or 20 thymines (T), adenines (A), or similar nucleotides (such as artificial nucleotides). In some embodiments, the noncomplementary region is a series of thymines (0-10 nucleotides). In some embodiments, there is no noncomplementary region present in the primer or methods.

In some embodiments, the target primer does not include a noncomplementary region.

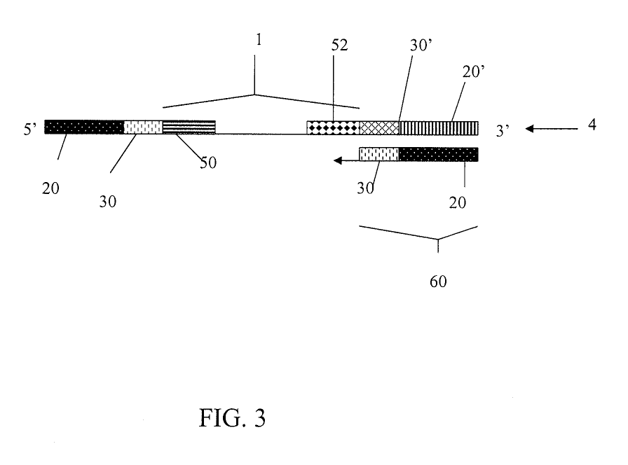

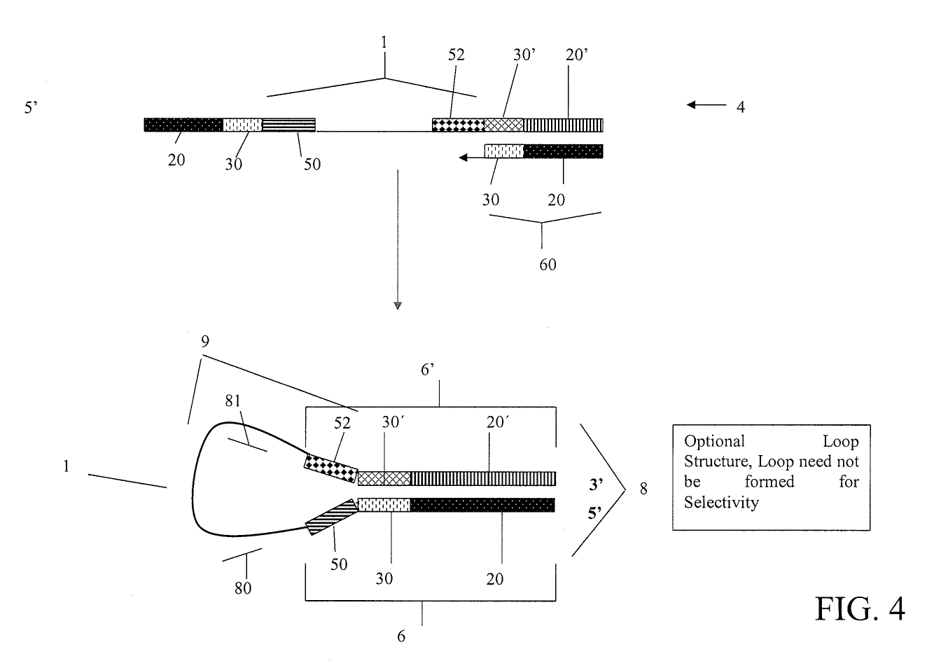

As will be appreciated by one of skill in the art, while the term “noncomplementary” can denote that the sequence does not significantly or functionally complement another sequence in a mixture, there will be sequences that can hybridize to the noncomplementary region. For example, in a double-extended target primer (FIG. 3) there is both the target primer and the target primer complement (of course, as the target primer and the target primer complement can include random regions, they need not be 100% complementary at the 3′ end, as shown in FIG. 4). Additionally, as noted above, in some embodiments, the amplification primer can also include a sequence that can hybridize to the noncomplementary region.

The term “does not effectively hybridize” denotes that the amount of hybridization that occurs is such that a significant reduction in primer dimerization or other forms of nonspecific hybridization occurs.

As used herein, the “target binding site” refers to a nucleic acid sequence, in the target nucleic acid sequence, where the 3′ target-specific portion of the target primer can or is configured to hybridize to. As will be appreciated by one of skill in the art, this section can be part of the target nucleic acid sequence and can therefore be gDNA or other nucleic acid sequences.

As used herein, the “extended target primer” refers to a nucleic acid sequence that has been extended from a target primer hybridized to a target binding site. The extended target primer can include the target primer, along with a sequence that is effectively complementary to a sequence that is contained within the target sequence. In some embodiments the extended target primer is linear. In some embodiments, the extended portion of the extended target primer (that is to become the longer double-extended primer or double extended linear primer) is at least 100 nucleotides in length. In some embodiments, this extended portion is at least 200 nucleotides in length. In some embodiments, this extended portion is not more than 10 kb in length. As will be appreciated by one of skill in the art, those double-extended primers that are to become the shorter double extended primer can be shorter than the above ranges. In addition, in some embodiments, other lengths are contemplated. As will be appreciated by one of skill in the art, the “extended target primer” will include a target primer; however, it will not need to serve as a primer itself.

As used herein, the “target primer complement” refers to a nucleic acid sequence that is the complement of the target primer (of course, the target primer complement need not be 100% complementary, as the primers can include degenerate or random regions). As will be appreciated by one of skill in the art, in some embodiments, the sequence of the target primer complement can still form a looped primer itself. Additionally, any universal region and/or noncomplementary region in the target primer complement will be complementary to the relevant section in the target primer. An example of a target primer complement can be found in FIG. 3, on the right hand side of sequence 4, including sections 20′, 30′, and 52. However, as noted above, a target primer complement need not have a 3′ target specific region that is complementary to the 3′ target specific region in the target primer (as these can be from different initial target primers).

As used herein, the “universal region complement” refers to a nucleic acid sequence that is the complement of the sequence in the universal region.

The term “double-extended target primer” refers to a nucleic acid sequence that has been formed by extending a second target primer that is hybridized to an extended first target primer. In other words, the nucleic acid sequence has been extended twice via target primers. In some embodiments, the term “double extended target primer” simply means that there is a nucleic acid sequence that includes a target primer, a target sequence, and a target primer complement; the method by which it is made is not relevant. In some embodiments, the term “double extended target primer” simply means that there is a nucleic acid sequence that includes a universal region, a target sequence, and a universal region complement; the method by which it is made is not relevant. As will be appreciated by one of skill in the art, the “double-extended target primer” can include a target primer and a target primer complement (or just a universal region and a universal region complement); however, it does not necessarily need to serve as a primer itself.

The “amplification primer” can be used for amplifying the double extended target primer. An example of such a primer is depicted in FIG. 3, as 60. In some embodiments, the amplification primer comprises or consists of the universal region 20. In some embodiments, the amplification primer comprises or consists of the universal region 20 and/or a noncomplementary region 30. In some embodiments, the amplification primer is a second target primer. In some embodiments, the amplification primer is not complementary to a first target primer. In some embodiments, the amplification primer has at least some of the same sequence as the target primer. In some embodiments, the amplification primer includes a sequence that is the same as the noncomplementary region. In some embodiments, the amplification primer includes a sequence that is the same as the universal region. As will be appreciated by one of skill in the art, the sequences need not be identical in all embodiments, as sequences that still selectively hybridize to the desired location can be employed as well. In some embodiments, the amplification primer is between 10-40 nucleotides long, such as a 30-mer. In some embodiments the amplification primer is 14 nucleotides long. In some embodiments, the amplification primer includes a “universal reverse primer,” which indicates that the sequence of the reverse primer can be used in a plurality of different reactions querying different target nucleic acid sequences, but that the amplification primer nonetheless can be the same sequence. In some embodiments, the amplification primer includes a tail region that is not complementary to the sequence that the rest of the primer hybridizes to.

The term “insert section,” “insert,” “capture section,” or “target section” refers to the section from one 3′ target specific region to a second 3′ target specific region, as shown in FIG. 4. In some embodiments, the insert section includes the 3′ target specific region as well; thus, the insert section includes 52 and 50 in FIG. 4, and is defined between 20 and 20′ and optionally 30 and 30′. As will be appreciated by one of skill in the art, in some embodiments, the insert section 9 can be looped, such as by the hybridization of the universal region and the universal region complement in a double-extended primer 8, as shown in FIG. 4, (e.g., the loop formed by the self-hybridization of the double-extended linear primer). However, in other embodiments, the insert section is not actually looped during various amplification steps (although they will be looped for the shorter insert sections, such as primer dimers, that are not to be amplified). As described in more detail below, even when not part of a looped structure, the length of the insert section or target section can still influence the amplification of the section. For example, shorter length insert sections will result in closer to zero order reaction kinetics between the universal region and its complement, while longer insert sections will increase the distance between the universal region and its complement, resulting in slower reaction kinetics. Thus, double extended target primers need not be looped in order to allow for selective amplification of longer insert sections over shorter insert sections. As will be appreciated by one of skill in the art, one can characterize the insert section as including some of the target primer sequence. Unless otherwise stated, “insert section” will include the region to which the target primer initially binds. Thus, a double extended target primer that is only a primer dimer, even if it includes nothing more than the random region of the linear primer, can still be characterized as “having” an insert section that is shorter than another double extended target primer. That is, an “insert section” does not have to include any target (or foreign) nucleic acid sequence and can simply be one or two random regions from the target primers.

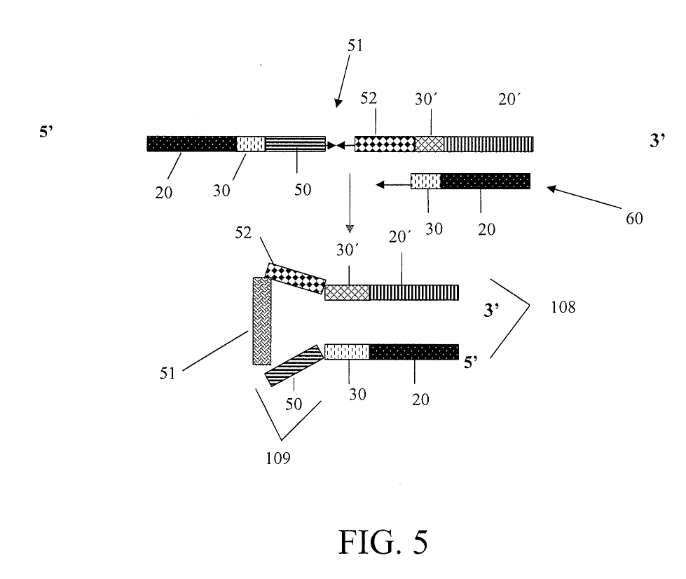

In some embodiments, the insert section 9 can include a significant portion of target nucleic acid sequence, as shown in FIG. 4, which can then be amplified. Alternatively, the insert section can contain an insignificant amount of target DNA 51 (such as when primer dimers occur or overly frequent priming occurs), such an embodiment is shown in FIG. 5. In some embodiments, the insignificant amount of DNA 51 will be no DNA, as such, the insert section is only 50 connected to 52. In other embodiments, a small amount of the target nucleic acid sequence in included 51. In some embodiments, the insert section for the double-extended target primer to be amplified is between 100 bp and 20 kb nucleotides in length.

The “capture stem” or “insert stem” denotes the section of the double-extended target primer that is self-hybridized. As will be appreciated by one of skill in the art, when the double extended target primer is simply a primer dimer, without any additional target nucleic acid sequence, the insert section will comprise the original linear primer sequences. As the structure can still be looped, there can still be unpaired nucleotides within the loop (although there need not be). Such primer dimer formations can be characterized as having an “empty insert section” or “no insert section”, as they contain no additional sequence, apart from the starting primers. Alternatively, such primer dimer formations can be characterized as having “no foreign insert section”, as they contain no additional sequence, apart from the starting primers; however, as they will still include the 3′ target specific regions, there can still be a sequence within the insert section, even though none of it is foreign

The term “insert amplification primer” refers to a primer that can be used to amplify the insert section. Generally, these primers are complementary to some section of the target nucleic acid sequence that is within the double-extended target primer. In some embodiments, the insert amplification primers are specific primers with known or knowable sequences. In some embodiments, numerous insert amplification primers will be employed as the specific sequence that has been amplified may not be known. In some embodiments, two or more insert amplification primers are used to amplify the insert sections. In some embodiments, each insert amplification primer (or paired set thereof) will be combined with the double-extended target primer in a separate reaction chamber (thus the amplified double-extended target primer will be divided between numerous reaction chambers). In other embodiments, the numerous insert amplification primers and the amplification reaction are performed in a single reaction chamber or are combined in some manner. In some embodiments, the insert amplification primers are degenerate primers. In some embodiments, the insert amplification primers are relatively short to allow for ease of amplification. In some embodiments, the insert amplification primers include universal bases.

The term “intramolecular hybridization” refers to an event or state in which at least a portion of a nucleic acid strand is hybridized to itself.

The terms “self-hybridizing” or “self-hybridized” refer to an event or state in which a portion of a nucleic acid strand is hybridized to another portion of itself. In general, the term is reserved for the effective hybridization of the universal region of the target primer to at least a portion of the universal region complement (which can be within a target primer complement) in a double extended target primer, e.g., as shown in FIG. 5. For example the universal region can be hybridized to the universal region complement.

The term “large enough to allow amplification” in reference to the insert section (or looped target section) denotes that, relative to other species of sequences in the reaction mixture, the larger size of the insert of the described species allows for greater or more efficient amplification. If an insert has a “significant portion of target DNA” it will be large enough to allow amplification. In some embodiments, the insert is between 200 bp and 10 kb or more nucleic acids in length. In some embodiments, the relative prevention is between a primer dimer (which comprises only the sequence of the target primer, e.g., a primer dimer) and a double extended target primer that includes at least one nucleotide in addition to the target primer.

The term “short enough to reduce the likelihood that amplification will occur” in reference to the insert section denotes that, relative to other species of sequences in the reaction mixture, the smaller size of the insert of the described species results in less and/or less efficient amplification compared to another species in the reaction mixture. If an insert has “an insignificant amount of target DNA” it is small enough to prevent or reduce the likelihood of amplification of the insert. In some embodiments, an insert that is short enough to reduce the likelihood that amplification will occur is between 1 and 200 nucleotides in length. In some embodiments, an insignificant amount of target DNA is from 1 to 200 nucleotides in length. As will be appreciated by one of skill in the art, as the target primer and primer complement can include a 3′ target specific region some amount of the sequence of the target nucleic acid can appear to be present, even in situations where simple primer dimerization has occurred (and thus no target has been incorporated into the structure). In some embodiments, the above two terms are defined relative to one another. As will be appreciated by one of skill in the art in light of the present disclosure, in some embodiments the size of the looped target section (or insert section) is being used to preferentially reduce the amplification of smaller regions of the target nucleic acid sequence compared to larger target nucleic acid sequences. Thus, in some embodiments, the “prevention” or “reduction” of the amplification of a first double-extended target primer over a second double-extended target primer results from the fact that the first has a shorter insert section compared to the second. In some embodiments, any difference in size of the insert section can result in the desired “reduction” or selective amplification, for example, the insert section in a first double extended primer can be 99-90, 90-80, 80-70, 70-60, 60-50, 50-40, 40-30, 30-20, 20-10, 10-5, 5-1, 1-0.1, 0.1-0.001% or less the size of the insert section in the second double-extended target primer. In some embodiments, the prevention or reduction is specific to the prevention of the amplification of primer dimers. In such embodiments, the insert section is included in the primer portions, as these are the only portions that make up the entire structure. As will be appreciated by one of skill in the art, for primer dimers, the insert section is part of the primers themselves, as no additional sequence need be added. Thus, in such embodiments, the insert section overlaps with the 3′ target specific region, the noncomplementary region, and/or the universal region. The insert section itself simply denotes the part that connects one primer to a previously separate primer, and can be part of one of the original primer sequences (e.g., the 3′ target specific region, the noncomplementary region and/or the universal region). In some embodiments, primer dimers (structures that result from two primers hybridizing to one another and being extended) “include” an insert short enough to reduce the likelihood of amplification. Thus, in some embodiments, primer dimers will be removed from subsequent amplification. In some embodiments, the insert section in a primer dimer will not be large enough to allow amplification of the structure or the looped section in the primer.

In some embodiments, relative prevention is between designated larger and smaller sections. In some embodiments, the relative prevention or reduction in likelihood is in comparison to the same sequence as the insert sequence, except that the sequence is not looped (e.g., same insert sections sequence, but no or insignificant amounts of the stem forming region). In some embodiments, the relative prevention is between a primer dimer (which comprises only the sequence of the target primer, e.g., a primer dimer) and a double extended target primer that includes at least one nucleotide in addition to the target primer.

As will be appreciated by one of skill in the art, in embodiments in which one is amplifying within a self-hybridized structure, at large enough lengths, the amplification in the insert section does not change significantly upon increasing the length of the nucleic acid sequence in the insert section. However, these sequences can still be preferentially amplified over double-extended target primers having shorter length insert sections. As noted below, in some embodiments, insert sections of at least 100 bp are generally used in order to have amplification in the loop. In embodiments in which SNP genotyping and gene dosage RT-PCR are employed, the length of the loops can be 100 bp longer, in order to allow spacing for two primers and probes (e.g., TAQMAN® probes). For some embodiments, such as capillary electrophoresis for sequencing applications, the insert sections can be 500 bp or longer. Insert sections of at least 500 bp can result in very efficient amplification in the loop. If longer loops are desired, the annealing time and/or extension time can be increased during PCR. In embodiments in which a self-hybridized structure is not formed for the longer double extended linear primer, then there need be no minimal size, as long as it is longer than the other double extended linear primer that the long double extended linear primer is to be amplified over.

As used herein, the term “identifying portion” refers to a moiety or moieties that can be used to identify a particular target primer species, and can refer to a variety of distinguishable moieties including zip-codes, a known number of nucleobases, and combinations thereof. In some embodiments, an identifying portion, or an identifying portion complement, can hybridize to a detector probe, thereby allowing detection of a target nucleic acid sequence in a decoding reaction. The terms “identifying portion complement” typically refers to at least one oligonucleotide that comprises at least one sequence of nucleobases that are at least substantially complementary to and hybridize with their corresponding identifying portion. In some embodiments, identifying portion complements serve as capture moieties for attaching at least one identifier portion and target nucleic acid sequence to at least one substrate; serve as “pull-out” sequences for bulk separation procedures; or both as capture moieties and as pull-out sequences (see for example O'Neil, et al., U.S. Pat. Nos. 6,638,760, 6,514,699, 6,146,511, and 6,124,092).

Typically, identifying portions and their corresponding identifying portion complements are selected to minimize: internal, self-hybridization; cross-hybridization with different identifying portion species, nucleotide sequences in a reaction composition, including but not limited to gDNA, different species of identifying portion complements, or target-specific portions of probes, and the like; but should be amenable to facile hybridization between the identifying portion and its corresponding identifying portion complement. Identifying portion sequences and identifying portion complement sequences can be selected by any suitable method, for example but not limited to, computer algorithms such as described in PCT Publication Nos. WO 96/12014 and WO 96/41011 and in European Publication No. EP 799,897; and the algorithm and parameters of SantaLucia (Proc. Natl. Acad. Sci. 95:1460-65 (1998)). Descriptions of identifying portions can be found in, among other places, U.S. Pat. No. 6,309,829 (referred to as “tag segment” therein); U.S. Pat. No. 6,451,525 (referred to as “tag segment” therein); U.S. Pat. No. 6,309,829 (referred to as “tag segment” therein); U.S. Pat. No. 5,981,176 (referred to as “grid oligonucleotides” therein); U.S. Pat. No. 5,935,793 (referred to as “identifier tags” therein); and PCT Publication No. WO 01/92579 (referred to as “addressable support-specific sequences” therein).

In some embodiments, the detector probe can hybridize to both the identifying portion as well as a sequence corresponding to the target nucleic acid sequence. In some embodiments, at least two identifying portion-identifying portion complement duplexes have melting temperatures that fall within a Δ Tₘ range (Tₘₐₓ-Tₘᵢₙ) of no more than 10° C. of each other. In some embodiments, at least two identifying portion-identifying portion complement duplexes have melting temperatures that fall within a ΔTₘ range of 5° C. or less of each other. In some embodiments, at least two identifying portion-identifying portion complement duplexes have melting temperatures that fall within a Δ Tₘ range of 2° C. or less of each other.

In some embodiments, at least one identifying portion or at least one identifying portion complement is used to separate the element to which it is bound from at least one other component of a ligation reaction composition, a digestion reaction composition, an amplified ligation reaction composition, or the like. In some embodiments, identifying portions are used to attach at least one ligation product, at least one ligation product surrogate, or combinations thereof, to at least one substrate. In some embodiments, at least one ligation product, at least one ligation product surrogate, or combinations thereof, comprise the same identifying portion. Examples of separation approaches include but are not limited to, separating a multiplicity of different element-identifying portion species using the same identifying portion complement, tethering a multiplicity of different element-identifying portion species to a substrate comprising the same identifying portion complement, or both. In some embodiments, at least one identifying portion complement comprises at least one label, at least one mobility modifier, at least one label binding portion, or combinations thereof. In some embodiments, at least one identifying portion complement is annealed to at least one corresponding identifying portion and, subsequently, at least part of that identifying portion complement is released and detected, see for example Published P.C.T. Application WO04/4634 to Rosenblum et al., and Published P.C.T. Application WO01/92579 to Wenz et al.

As will be appreciated by one of skill in the art, while the presently disclosed target primers can include an identifying portion, it need not be included and is not included in some embodiments. In some embodiments, the target primer includes an identifying portion as well as the noncomplementary region. Is some embodiments, the identifying portion is not the same as the noncomplementary region. In some embodiments, an identifying portion is not included in a target primer.

As used herein, the term “extension reaction” refers to an elongation reaction in which the 3′ target specific portion of a target primer is extended to form an extension reaction product comprising a strand complementary to a target nucleic acid sequence. In some embodiments, the target nucleic acid sequence is a gDNA molecule or fragment thereof. In some embodiments, the target nucleic acid sequence is a short DNA molecule and the extension reaction comprises a polymerase and results in the synthesis of a 2^{nd} strand of DNA. In some embodiments, the consolidation of the extension reaction and a subsequent amplification reaction is further contemplated by the present teachings.

As used herein, the term “primer portion” refers to a region of a polynucleotide sequence that can serve directly, or by virtue of its complement, as the template upon which a primer can anneal for any of a variety of primer nucleotide extension reactions known in the art (for example, PCR). It will be appreciated by those of skill in the art that when two primer portions are present on a single polynucleotide, the orientation of the two primer portions is generally different. For example, one PCR primer can directly hybridize to a first primer portion, while another PCR primer can hybridize to the complement of the second primer portion. In some embodiments, “universal” primers and primer portions as used herein are generally chosen to be as unique as possible given the particular assays and sequences involved to ensure specificity of the assay. However, as will be appreciated by one of skill in the art, when a noncomplementary region is employed, the need for uniqueness with regard to the universal region is greatly diminished if not removed completely.

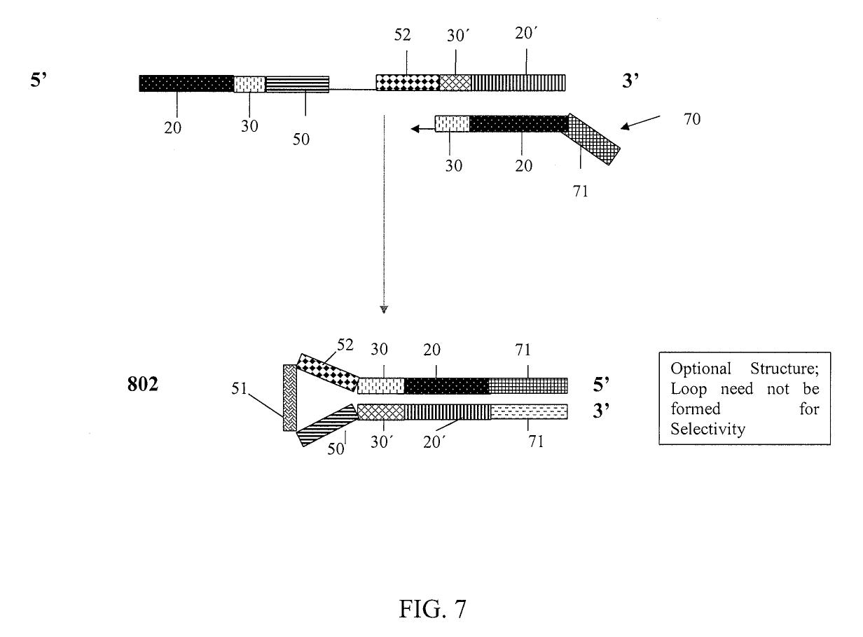

The term “tail region” of a primer denotes a section at the 5′ end of a primer sequence. In some embodiments this section can hybridize to part of a target sequence or priming site (e.g., such that the entire primer is hybridized to a target sequence or priming site). In some embodiments, the tail region has a sequence that is not complementary to the nucleic acid sequence that the remaining portion of the primer has hybridized to (e.g., the 5′ end is not hybridized to a priming site while the rest of the primer can hybridize). In some embodiments, primers having different tail regions are used so as to allow for a sequence difference to be made at each end of the nucleic acid sequence (e.g., as shown in FIG. 7). Such a tail region can be denoted as a “noncomplementary tail region” or a second tail region, wherein the second tail region is different from the first. In some embodiments, the tail portion can include a zip-code, which can allow for the identification or tracking of the molecule associated with the zip-code. In some embodiments, the tail portion of the forward primer is between 5-8 nucleotides long. As will be appreciated by one of skill in the art, the length of the tail can determine the stability of the stem loop. If primer dimers are not a significant problem, the tail can be, for example, as large as a 20-mer to allow for the incorporation of forward and reverse primers for sequencing reactions that require two different primers. In some embodiments, one can reduce potential primer-dimer formation from carry over random primers by using tails that are less than 5-8 nucleotides in length. In some embodiments, a noncomplementary tail region is not used.

In some embodiments, the tail portion of the forward primer is 6 nucleotides long. Those in the art will appreciate that forward primer tail portion lengths shorter than 5 nucleotides and longer than 8 nucleotides can be identified in the course of routine methodology and without undue experimentation, and that such shorter and longer forward primer tail portion lengths are contemplated by the present teachings.

The term “upstream” as used herein takes on its customary meaning in molecular biology, and refers to the location of a region of a polynucleotide that is on the 5′ side of a “downstream” region. Correspondingly, the term “downstream” refers to the location of a region of a polynucleotide that is on the 3′ side of an “upstream” region.

As used herein, the term “hybridization” refers to the complementary base-pairing interaction of one nucleic acid with another nucleic acid that results in formation of a duplex, triplex, or other higher-ordered structure, and is used herein interchangeably with “annealing.” Typically, the primary interaction is base specific, e.g., A/T and G/C, by Watson/Crick and Hoogsteen-type hydrogen bonding. Base-stacking and hydrophobic interactions can also contribute to duplex stability. Conditions for hybridizing detector probes and primers to complementary and substantially complementary target sequences are well known, e.g., as described in Nucleic Acid Hybridization, A Practical Approach, B. Hames and S. Higgins, eds., IRL Press, Washington, D.C. (1985) and J. Wetmur and N. Davidson, Mol. Biol. 31:349 et seq. (1968). In general, whether such annealing takes place is influenced by, among other things, the length of the polynucleotides and the complementarity, the pH, the temperature, the presence of mono- and divalent cations, the proportion of G and C nucleotides in the hybridizing region, the viscosity of the medium, and the presence of denaturants. Such variables influence the time required for hybridization. Thus, the preferred annealing conditions will depend upon the particular application. Such conditions, however, can be routinely determined by the person of ordinary skill in the art without undue experimentation. It will be appreciated that complementarity need not be perfect; there can be a small number of base pair mismatches that will minimally interfere with hybridization between the target sequence and the single stranded nucleic acids of the present teachings. However, if the number of base pair mismatches is so great that no hybridization can occur under minimally stringent conditions then the sequence is generally not a complementary target sequence. Thus, complementarity herein is meant that the probes or primers are sufficiently complementary to the target sequence to hybridize under the selected reaction conditions to achieve the ends of the present teachings. Something is “configured to hybridize” when its sequence (e.g., structure) allows hybridization through base specific, e.g., A/T and G/C, by Watson/Crick and Hoogsteen-type hydrogen bonding.

As used herein, the term “amplifying” refers to any method by which at least a part of a target nucleic acid sequence, target nucleic acid sequence surrogate, or combinations thereof, is reproduced, typically in a template-dependent manner, including without limitation, a broad range of techniques for amplifying nucleic acid sequences, either linearly or exponentially. Exemplary means for performing an amplifying step include, but are not limited to, oligonucleotide ligation assay (OLA), ligase chain reaction (LCR), ligase detection reaction (LDR), ligation followed by Q-replicase amplification, PCR, primer extension, strand displacement amplification (SDA), hyperbranched strand displacement amplification, multiple displacement amplification (MDA), nucleic acid strand-based amplification (NASBA), two-step multiplexed amplifications, rolling circle amplification (RCA) and the like, including multiplex versions or combinations thereof, for example but not limited to, OLA/PCR, PCR/OLA, LDR/PCR, PCR/PCR/LDR, PCR/LDR, LCR/PCR, PCR/LCR (also known as combined chain reaction-CCR), and the like. Descriptions of such techniques can be found in, among other places, Sambrook et al. Molecular Cloning, 3.sup.rd Edition; Ausbel et al.; PCR Primer: A Laboratory Manual, Diffenbach, Ed., Cold Spring Harbor Press (1995); The Electronic Protocol Book, Chang Bioscience (2002), Msuih et al., J. Clin. Micro. 34:501-07 (1996); The Nucleic Acid Protocols Handbook, R. Rapley, ed., Humana Press, Totowa, N.J. (2002); Abramson et al., Curr Opin Biotechnol. 1993 February; 4(1):41-7, U.S. Pat. No. 6,027,998; U.S. Pat. No. 6,605,451, Barany et al., PCT Publication No. WO 97/31256; Wenz et al., PCT Publication No. WO 01/92579; Day et al., Genomics, 29(1): 152-162 (1995), Ehrlich et al., Science 252:1643-50 (1991); Innis et al., PCR Protocols: A Guide to Methods and Applications, Academic Press (1990); Favis et al., Nature Biotechnology 18:561-64 (2000); and Rabenau et al., Infection 28:97-102 (2000); Belgrader, Barany, and Lubin, Development of a Multiplex Ligation Detection Reaction DNA Typing Assay, Sixth International Symposium on Human Identification, 1995 (available on the world wide web at: promega.com/geneticidproc/ussymp6proc/blegrad.html); LCR Kit Instruction Manual, Cat. #200520, Rev. #050002, Stratagene, 2002; Barany, Proc. Natl. Acad. Sci. USA 88:188-93 (1991); Bi and Sambrook, Nucl. Acids Res. 25:2924-2951 (1997); Zirvi et al., Nucl. Acid Res. 27:e40i-viii (1999); Dean et al., Proc Natl Acad Sci USA 99:5261-66 (2002); Barany and Gelfand, Gene 109:1-11 (1991); Walker et al., Nucl. Acid Res. 20:1691-96 (1992); Polstra et al., BMC Inf. Dis. 2:18-(2002); Lage et al., Genome Res. 2003 February; 13(2):294-307, and Landegren et al., Science 241:1077-80 (1988), Demidov, V., Expert Rev Mol. Diagn. 2002 November; 2(6):542-8., Cook et al., J Microbiol Methods. 2003 May; 53(2):165-74, Schweitzer et al., Curr Opin Biotechnol. 2001 February; 12(1):21-7, U.S. Pat. No. 5,830,711, U.S. Pat. No. 6,027,889, U.S. Pat. No. 5,686,243, Published P.C.T. Application WO0056927A3, and Published P.C.T. Application WO9803673A1. In some embodiments, newly-formed nucleic acid duplexes are not initially denatured, but are used in their double-stranded form in one or more subsequent steps. An extension reaction is an amplifying technique that comprises elongating a target primer that is annealed to a template in the 5′ to 3′ direction using an amplifying means such as a polymerase and/or reverse transcriptase.

According to some embodiments, with appropriate buffers, salts, pH, temperature, and nucleotide triphosphates, including analogs thereof, i.e., under appropriate conditions, a polymerase incorporates nucleotides complementary to the template strand starting at the 3′-end of an annealed target primer, to generate a complementary strand. In some embodiments, the polymerase used for extension lacks or substantially lacks 5′ exonuclease activity. In some embodiments of the present teachings, unconventional nucleotide bases can be introduced into the amplification reaction products and the products treated by enzymatic (e.g., glycosylases) and/or physical-chemical means in order to render the product incapable of acting as a template for subsequent amplifications. In some embodiments, uracil can be included as a nucleobase in the reaction mixture, thereby allowing for subsequent reactions to decontaminate carryover of previous uracil-containing products by the use of uracil-N-glycosylase (see for example Published P.C.T. Application WO9201814A2).

In some embodiments of the present teachings, any of a variety of techniques can be employed prior to amplification in order to facilitate amplification success, as described for example in Radstrom et al., Mol. Biotechnol. 2004 February; 26(2):13346. In some embodiments, amplification can be achieved in a self-contained integrated approach comprising sample preparation and detection, as described for example in U.S. Pat. Nos. 6,153,425 and 6,649,378. Reversibly modified enzymes, such as, but not limited to, those described in U.S. Pat. No. 5,773,258, are also within the scope of the disclosed teachings. The present teachings also contemplate various uracil-based decontamination strategies, wherein for example uracil can be incorporated into an amplification reaction, and subsequent carry-over products removed with various glycosylase treatments (see for example U.S. Pat. No. 5,536,649, and U.S. Provisional Application 60/584,682 to Andersen et al.). Those in the art will understand that any protein with the desired enzymatic activity can be used in the disclosed methods and kits. Descriptions of DNA polymerases, including reverse transcriptases, uracil N-glycosylase, and the like, can be found in, among other places, Twyman, Advanced Molecular Biology, BIOS Scientific Publishers, 1999; Enzyme Resource Guide, rev. 092298, Promega, 1998; Sambrook and Russell; Sambrook et al.; Lehninger; PCR: The Basics; and Ausbel et al.

As used herein, the term “detector probe” refers to a molecule used in an amplification reaction, typically for quantitative or real-time PCR analysis, as well as end-point analysis. Such detector probes can be used to monitor the amplification of the target nucleic acid sequence. In some embodiments, detector probes present in an amplification reaction are suitable for monitoring the amount of amplicon(s) produced as a function of time. Such detector probes include, but are not limited to, the 5′-exonuclease assay (TAQMAN® probes described herein (see also U.S. Pat. No. 5,538,848) various stem-loop molecular beacons (see e.g., U.S. Pat. Nos. 6,103,476 and 5,925,517 and Tyagi and Kramer, 1996, Nature Biotechnology 14:303-308), stemless or linear beacons (see, e.g., WO 99/21881), PNA Molecular Beacons™ (see, e.g., U.S. Pat. Nos. 6,355,421 and 6,593,091), linear PNA beacons (see, e.g., Kubista et al., 2001, SPIE 4264:53-58), non-FRET probes (see, e.g., U.S. Pat. No. 6,150,097), Sunrise®/Amplifluor™ probes (U.S. Pat. No. 6,548,250), stem-loop and duplex Scorpion probes (Solinas et al., 2001, Nucleic Acids Research 29:E96 and U.S. Pat. No. 6,589,743), bulge loop probes (U.S. Pat. No. 6,590,091), pseudo knot probes (U.S. Pat. No. 6,589,250), cyclicons (U.S. Pat. No. 6,383,752), MGB Eclipse™ probe (Epoch Biosciences), hairpin probes (U.S. Pat. No. 6,596,490), peptide nucleic acid (PNA) light-up probes, self-assembled nanoparticle probes, and ferrocene-modified probes described, for example, in U.S. Pat. No. 6,485,901; Mhlanga et al., 2001, Methods 25:463-471; Whitcombe et al., 1999, Nature Biotechnology. 17:804-807; Isacsson et al., 2000, Molecular Cell Probes. 14:321-328; Svanvik et al., 2000, Anal Biochem. 281:26-35; Wolffs et al., 2001, Biotechniques 766:769-771; Tsourkas et al., 2002, Nucleic Acids Research. 30:4208-4215; Riccelli et al., 2002, Nucleic Acids Research 30:4088-4093; Zhang et al., 2002 Shanghai. 34:329-332; Maxwell et al., 2002, J. Am. Chem. Soc. 124:9606-9612; Broude et al., 2002, Trends Biotechnol. 20:249-56; Huang et al., 2002, Chem. Res. Toxicol. 15:118-126; and Yu et al., 2001, J. Am. Chem. Soc 14:11155-11161. Detector probes can also comprise quenchers, including without limitation black hole quenchers (Biosearch), Iowa Black (IDT), QSY quencher (Molecular Probes), and Dabsyl and Dabcel sulfonate/carboxylate Quenchers (Epoch). Detector probes can also comprise two probes, wherein for example a fluor is on one probe, and a quencher is on the other probe, wherein hybridization of the two probes together on a target quenches the signal, or wherein hybridization on the target alters the signal signature via a change in fluorescence. Detector probes can also comprise sulfonate derivatives of fluorescenin dyes with SO₃ instead of the carboxylate group, phosphoramidite forms of fluorescein, phosphoramidite forms of CY 5 (commercially available for example from Amersham). In some embodiments, interchelating labels are used such as ethidium bromide, SYBR® Green I (Molecular Probes), and PicoGreen® (Molecular Probes), thereby allowing visualization in real-time, or end point, of an amplification product in the absence of a detector probe. In some embodiments, real-time visualization can comprise the use of both an intercalating detector probe and a sequence-based detector probe. In some embodiments, the detector probe is at least partially quenched when not hybridized to a complementary sequence in the amplification reaction, and is at least partially unquenched when hybridized to a complementary sequence in the amplification reaction. In some embodiments, the detector probes of the present teachings have a Tm of 63-69° C., though it will be appreciated that with guidance provided by the present teachings, routine experimentation can result in detector probes with other Tms. In some embodiments, probes can further comprise various modifications such as a minor groove binder (see for example U.S. Pat. No. 6,486,308) to further provide desirable thermodynamic characteristics. In some embodiments, detector probes can correspond to identifying portions or identifying portion complements.

The term “corresponding” as used herein refers to a specific relationship between the elements to which the term refers. Some non-limiting examples of “corresponding” include, but are not limited to: a target primer that corresponds to a target nucleic acid sequence, and vice versa; or a forward primer that corresponds to a target nucleic acid sequence, and vice versa. In some cases, the corresponding elements can be complementary. In some cases, the corresponding elements are not complementary to each other, but one element can be complementary to the complement of another element.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

As used herein, the term “reaction vessel” or “reaction chamber” generally refers to any container in which a reaction can occur in accordance with the present teachings. In some embodiments, a reaction vessel can be an eppendorf tube or other container of the sort in common use in modern molecular biology laboratories. In some embodiments, a reaction vessel can be a well in a microtitre plate, a spot on a glass slide, or a well in an Applied Biosystems TaqMan Low Density Array for gene expression (formerly MicroCard™). A plurality of reaction vessels can reside on the same support. In some embodiments, lab-on-a-chip like devices, available for example from Caliper and Fluidgm, can provide for reaction vessels. In some embodiments, various microfluidic approaches as described in U.S. Provisional Application 60/545,674 to Wenz et al., can be employed. It will be recognized that a variety of reaction vessels are available in the art and within the scope of the present teachings.

As used herein, the term “detection” refers to a way of determining the presence and/or quantity and/or identity of a target nucleic acid sequence. In some embodiments the sequence to be detected is known. Thus, in some embodiments, detection occurs by determining if the target nucleic acid sequence comprises or consists of a known nucleic acid sequence, gene, etc. In some embodiments, the sequence to be detected is not known prior to the experiment. In such embodiments, the target nucleic acid sequence is amplified and sequenced. The sequencing of the target nucleic acid can be characterized as “detecting” the target nucleic acid. The target nucleic acid sequence to be sequenced can be known or unknown prior to its sequencing. Thus, in some embodiments, a target nucleic acid is sequenced to determine if a specific sequence or gene is present in a sample, and/or determine what specific variant is present. In some embodiments, a target nucleic acid is sequenced to determine the sequences of the genes or nucleic acid sequences themselves (e.g., the sequence and/or identity of the target nucleic acid sequence is not known prior to sequencing).

In some embodiments employing a donor moiety and signal moiety, one can use certain energy-transfer fluorescent dyes for detection. Certain nonlimiting exemplary pairs of donors (donor moieties) and acceptors (signal moieties) are illustrated, e.g., in U.S. Pat. Nos. 5,863,727; 5,800,996; and 5,945,526. Use of some combinations of a donor and an acceptor have been called FRET (Fluorescent Resonance Energy Transfer). In some embodiments, fluorophores that can be used as signaling probes include, but are not limited to, rhodamine, cyanine 3 (Cy 3), cyanine 5 (Cy 5), fluorescein, VIC®, LIZ®, TAMRA™ (carboxytetramethylrhodamine, succinimidyl ester), 5-FAM™ (5-carboxyfluorescein), 6-FAM™ (6-carboxyfluorescein), and Texas Red (Molecular Probes). (VIC®, LIZ®, TAMRA™, 5-FAM™, and 6-FAM™ all available from Applied Biosystems, Foster City, Calif.). In other embodiments, detection reagents including, but not limited to, SYBR® green dye or BEBO dye can be employed for detection.

In some embodiments, the amount of detector probe that gives a fluorescent signal in response to an excited light typically relates to the amount of nucleic acid produced in the amplification reaction. Thus, in some embodiments, the amount of fluorescent signal is related to the amount of product created in the amplification reaction. In such embodiments, one can therefore measure the amount of amplification product by measuring the intensity of the fluorescent signal from the fluorescent indicator.

According to some embodiments, one can employ an internal standard to quantify the amplification product indicated by the fluorescent signal. See, e.g., U.S. Pat. No. 5,736,333. Devices have been developed that can perform a thermal cycling reaction with compositions containing a fluorescent indicator, emit a light beam of a specified wavelength, read the intensity of the fluorescent dye, and display the intensity of fluorescence after each cycle. Devices comprising a thermal cycler, light beam emitter, and a fluorescent signal detector, have been described, e.g., in U.S. Pat. Nos. 5,928,907; 6,015,674; and 6,174,670, and include, but are not limited to the ABI Prism® 7700 Sequence Detection System (Applied Biosystems, Foster City, Calif.), the ABI GeneAmp® 5700 Sequence Detection System (Applied Biosystems, Foster City, Calif.), the ABI GeneAmp® 7300 Sequence Detection System (Applied Biosystems, Foster City, Calif.), and the ABI GeneAmp® 7500 Sequence Detection System (Applied Biosystems). In some embodiments, each of these functions can be performed by separate devices. For example, if one employs a Q-beta replicase reaction for amplification, the reaction does not need to take place in a thermal cycler, but could include a light beam emitted at a specific wavelength, detection of the fluorescent signal, and calculation and display of the amount of amplification product. In some embodiments, combined thermal cycling and fluorescence detecting devices can be used for precise quantification of target nucleic acid sequences in samples. In some embodiments, fluorescent signals can be detected and displayed during and/or after one or more thermal cycles, thus permitting monitoring of amplification products as the reactions occur in “real time.” In some embodiments, one can use the amount of amplification product and number of amplification cycles to calculate how much of the target nucleic acid sequence was in the sample prior to amplification.

In some embodiments, one can simply monitor the amount of amplification product after a predetermined number of cycles sufficient to indicate the presence of the target nucleic acid sequence in the sample. One skilled in the art can easily determine, for any given sample type, primer sequence, and reaction condition, how many cycles are sufficient to determine the presence of a given target nucleic acid sequence. As used herein, determining the presence of a target can comprise identifying it, as well as optionally quantifying it. In some embodiments, the amplification products can be scored as positive or negative as soon as a given number of cycles is complete. In some embodiments, the results can be transmitted electronically directly to a database and tabulated. Thus, in some embodiments, large numbers of samples can be processed and analyzed with less time and labor when such an instrument is used.

In some embodiments, different detector probes can distinguish between different target nucleic acid sequences. A non-limiting example of such a probe is a 5′-nuclease fluorescent probe, such as a TaqMan® probe molecule, wherein a fluorescent molecule is attached to a fluorescence-quenching molecule through an oligonucleotide link element. In some embodiments, the oligonucleotide link element of the 5′-nuclease fluorescent probe binds to a specific sequence of an identifying portion or its complement. In some embodiments, different 5′-nuclease fluorescent probes, each fluorescing at different wavelengths, can distinguish between different amplification products within the same amplification reaction. For example, in some embodiments, one could use two different 5′-nuclease fluorescent probes that fluoresce at two different wavelengths (WL_{A} and WL_{B}) and that are specific to two different stem regions of two different extension reaction products (A′ and B′, respectively). Amplification product A′ is formed if target nucleic acid sequence A is in the sample, and amplification product B′ is formed if target nucleic acid sequence B is in the sample. In some embodiments, amplification product A′ and/or B′ can form even if the appropriate target nucleic acid sequence is not in the sample, but such occurs to a measurably lesser extent than when the appropriate target nucleic acid sequence is in the sample. After amplification, one can determine which specific target nucleic acid sequences are present in the sample based on the wavelength of signal detected and their intensity. Thus, if an appropriate detectable signal value of only wavelength WL_{A} is detected, one would know that the sample includes target nucleic acid sequence A, but not target nucleic acid sequence B. If an appropriate detectable signal value of both wavelengths WL_{A} and WL_{B} are detected, one would know that the sample includes both target nucleic acid sequence A and target nucleic acid sequence B.

In some embodiments, detection can occur through any of a variety of mobility dependent analytical techniques based on differential rates of migration between different analyte species. Exemplary mobility-dependent analysis techniques include electrophoresis, chromatography, mass spectroscopy, sedimentation, e.g., gradient centrifugation, field-flow fractionation, multi-stage extraction techniques, and the like. In some embodiments, mobility probes can be hybridized to amplification products, and the identity of the target nucleic acid sequence determined via a mobility dependent analysis technique of the eluted mobility probes, as described for example in Published P.C.T. Application WO04/46344 to Rosenblum et al., and WO01/92579 to Wenz et al. In some embodiments, detection can be achieved by various microarrays and related software such as the Applied Biosystems Array System with the Applied Biosystems 1700 Chemiluminescent Microarray Analyzer and other commercially available array systems available from Affymetrix, Agilent, illumina, and Amersham Biosciences, among others (see also Gerry et al., J. Mol. Biol. 292:251-62, 1999; De Bellis et al., Minerva Biotec 14:247-52, 2002; and Stears et al., Nat. Med. 9:14045, including supplements, 2003). It will also be appreciated that detection can comprise reporter groups that are incorporated into the reaction products, either as part of labeled primers or due to the incorporation of labeled dNTPs during an amplification, or attached to reaction products, for example but not limited to, via hybridization tag complements comprising reporter groups or via linker arms that are integral or attached to reaction products. Detection of unlabeled reaction products, for example using mass spectrometry, is also within the scope of the current teachings.

The term “anneal” as used herein refers to the base-pairing interaction of one polynucleotide with another polynucleotide that results in the formation of a duplex or other higher-ordered structure. The primary interaction is base specific, i.e., A/T and G/C, by Watson/Crick and Hoogsteen-type hydrogen bonding.

The term “real-time analysis” refers to periodic monitoring during PCR. Certain systems such as the ABI 7700 and 7900HT Sequence Detection Systems (Applied Biosystems, Foster City, Calif.) conduct monitoring during each thermal cycle at a pre-determined or user-defined point. Real-time analysis of PCR with FRET probes measures fluorescent dye signal changes from cycle-to-cycle, preferably minus any internal control signals.

The term “5′-nuclease analysis” or “5′-nuclease assay” when used herein refers to “real-time analysis” for quantification of the amount of DNA amplified in a particular PCR reaction. TAQMAN® analysis is an example of such “5′-nuclease analysis” (a commercially available PCR kit). “5′-nuclease analysis” involves the use of a fluorogenic oligonucleotide probe to which a reporter dye and a quencher dye are attached. During amplification of a nucleotide sequence using a forward and reverse primer, the probe anneals to the target of interest between the forward and reverse primer sites. During extension, the probe is cleaved by the 5′-nuclease activity of the DNA polymerase. As the cleavage separates the reporter dye from the quencher dye, the reporter dye's fluorescence increases which can be detected and quantitated. Real-time analysis of PCR with 5′-nuclease assay involves FRET probes that can be displayed by plotting the logarithmic change in detected fluorescence (ΔRn) versus the cycle number. The cycle within the PCR protocol at which the change in fluorescence (ΔRn) rises above a threshold value is denoted as C_{T}. The C_{T} cycle is approximately the cycle at which amplification of target becomes exponential. A relatively low C_{T} value indicates efficient detection of amplicon. The threshold cycle is highly correlated to the amount of copy number, or amount of target nucleic acid sequence present in the sample, as well as the efficiency of amplification. The effects of primer constitution, e.g. length, sequence, mismatches, analogs, can be conveniently screened and quantitated by measurement of C_{T} values during real-time analysis of PCR. In some embodiments, the sequences within the insert sections can be detected and/or amplified via a TAQMAN® assay or similar assay.

The term “multiple displacement amplification” refers to a non-PCR based amplification process involving annealing primers to denatured nucleic acid sequences, followed by strand-displacement synthesis at a relatively constant temperature. While the temperature can vary throughout the process, the temperature does not increase to such an extent as to result in meaningful amount of melting to occur (and thus is functionally isothermal, in contrast to PCR). A key feature of this process is the displacement of intervening primers during amplification. This allows multiple copies of a nested set of the target nucleic acid sequence to be synthesized in a short period of time. Methods for a MDA are known in the art, and are described in various sources, for example, Dean, et al., “Comprehensive human genome amplification using multiple displacement amplification,” PNAS, vol. 99: 8 (2002); and U.S. Pat. No. 6,124,120 (Lizardi, et al., 2000), both of which are incorporated herein by reference in their entireties.

“Polymerase chain reaction” or “PCR” as used herein, refers to a method in the art for amplification of a nucleic acid. The method can involve introducing a molar excess of two or more extendable oligonucleotide primers to a reaction mixture comprising the desired target sequence(s), where the primers hybridize to opposite strands of the double stranded target sequence. The reaction mixture is subjected to a program of thermal cycling in the presence of a DNA polymerase, resulting in the amplification of the desired target sequence flanked by the oligonucleotide primers. The oligonucleotide primers prime multiple sequential rounds of DNA synthesis, each round of synthesis is typically separated by a melting and re-annealing step. Methods for a wide variety of PCR applications are widely known in the art, and are described in many sources, for example, Ausubel et al. (eds.), Current Protocols in Molecular Biology, Section 15, John Wiley & Sons, Inc., New York (1994). This technique is distinct from MDA in that PCR involves meaningful changes in temperature, which help drive the amplification process, while MDA occurs under isothermal conditions.

“In silico PCR” when used herein refers to a computer-conducted method for predicting the size and probability of amplification of a nucleotide sequence using a particular set of primers. The method involves searching a DNA database for exact matches to the primer sequences and further for sequences having the correct order, orientation, and spacing to allow priming of amplification of a nucleotide sequence of a predicted size.

“Tm” as used herein, refers to the melting temperature (temperature at which 50% of the oligonucleotide is a duplex) of the oligonucleotide calculated using the nearest-neighbor thermodynamic values of Breslauer et al. (Proc. Natl. Acad. Sci. USA 83:3746 3750, 1986) for DNA and Freier et al. (Proc. Natl. Acad. Sci. USA 83:9373 9377, 1986) for RNA.

As will be appreciated by one of skill in the art, the above definitions occasionally describe various embodiments that can also be used, in some embodiments, with the variously defined parts or steps. Unless indicated, these various embodiments are not required or part of the actual definitions and have been included for additional general context and for further description of the various contemplated embodiments.

Aspects of the present teachings can be further understood in light of the following description and examples, which should not be construed as limiting the scope of the present teachings in any way.

### Linear Primers

FIGS. 1A and 1B depict one embodiment of a target primer, in particular a linear primer 6, and an embodiment of its use. The linear primer can include a 3′ target specific region 50, a universal region 20, and optionally a noncomplementary region 30.

As described in detail below, and outlined in FIG. 1B, in some embodiments, the linear primer can be used to initiate priming as desired (e.g., via a random or degenerate priming region), while still including a universal and/or a noncomplementary region in the primer. Moreover, this can be achieved with a reduced risk of nonspecific or primer-dimer interactions occurring.

In some embodiments, such as the one depicted in FIG. 1B, the use of the linear primer to amplify sections of a target sequence allows one to place complementary sequences on either end of the amplified target nucleic acid sequence. As noted below, the addition of these complementary sequences allow for the selective amplification of the target nucleic acid sequences.

The first step depicted in FIG. 1B is the addition of a linear primer (6 depicted in FIG. 1A) to a solution that includes the target nucleic acid sequence or sequences that are to be amplified 110 or in which a target is to be identified, if present. Conditions are selected such that the linear primer hybridizes to the target sequence 120. The linear primer is then extended along the target sequence to form an extended linear primer 130. One can then allow a linear primer (the same degenerate linear primer, an identical linear primer, or a different linear primer, as long as the same universal region is present) to hybridize to the extended linear primer 140. Then one can extend the linear primer along the extended linear primer to form a double-extended linear primer 150. In various embodiments, the linear primers can have identical sequences; can have identical sequences apart from the 3′ target specific region; can have different sequences, apart from the noncomplementary region; or can have different sequences.

In some embodiments, some or all of steps 110-150 can be repeated as desired. In some embodiments, some or all of steps 110-150 can be repeated as desired prior to proceeding to step 160. Following step 150, one can optionally amplify the double-extended linear primer using an amplification primer 160. The amplification primer will have a sequence that will hybridize to a sequence that is complementary to the universal region on the primer (e.g., the amplification primer can have a sequence that is or is a part of the universal region) and, optionally (if necessary), a sequence that will hybridize to a sequence that is complementary to the noncomplementary region (e.g., is or is part of the noncomplementary region). As will be appreciated by one of skill in the art, in some embodiments, only one of these regions will be present.

In some embodiments, one can then allow the shorter double-extended linear primer to self-hybridize 170. In some embodiments, one can allow both the short and the long double-extended linear primers to self-hybridize. This self-hybridized population can then be used in the selective amplification of large insert sections over relatively small insert sections 180 (depicted in FIGS. 4 and 5). Thus, in some embodiments, the use of the linear primer described above results in a self-hybridized population that allows for the selective amplification of larger sections of target nucleic acid sequences over smaller sections of target nucleic acid sequences contained within the self-hybridized structures. In some embodiments an initial reverse transcription step can be performed or a cleaning step can be included, for example as described in the following sections.

While the self-hybridized structure can be used to help select a larger insert section (or insert sections) over smaller insert sections, the larger double extended linear primer need not assume a looped configuration. For example, in some embodiments, the self-hybridized structure is only formed for the shorter insert sections. Thus, in some embodiments, selective amplification of longer insert sections over shorter insert sections (including primer dimers) occurs without the formation of a self-hybridized structure for the longer double extended linear primer. Without intending to be limited by theory, it is understood that because a shorter insert sections will mean that there is less distance between the linear primer and the linear primer complement, that these short double extended linear primers will self hybridize faster than double extended linear primers with larger insert sections. Similarly, the larger double-extended linear primers will have more distance between the linear primer and its complement and thus it can take longer for the primer and its complement to self-hybridize. Thus, in some embodiments, it is the faster ability of the double extended linear primers having shorter insert sections to self-hybridize, and thus take themselves out of a reaction, that allows for the selective amplification of the double extended linear primers having the longer insert sections over the shorter (or no foreign) insert sections. Thus, in some embodiments, the longer or long insert section is not in a looped configuration during the selective amplification.

### Loopable Primers

FIG. 1C depicts one embodiment of a target primer 106, in particular a loopable primer. The loopable primer can include a 3′ target specific region 50, a first loop-forming region 10, a second loop forming-region 10′, a universal region 20 and, optionally, a noncomplementary region 30.

As described in detail below, and outlined in FIG. 1D, in some embodiments, the loopable primer can be used to initiate priming as desired (e.g., via a random or degenerate priming region), while still maintaining the ability to include a universal and a noncomplementary region in the primer. Moreover, this can be achieved with a reduced risk of nonspecific or primer-dimer interactions occurring.

In some embodiments, such as the one depicted in FIG. 1D, the use of the loopable primer to amplify sections of a target sequence allows one to place complementary sequences on either end of the amplified target nucleic acid sequence. As these sequences are complementary, they can hybridize together forming a looped structure (described as a self-hybridized double-extended loopable primer). As noted herein, subsequent amplification of the self-hybridized double-extended loopable primer will depend upon the size of the insert section. When relatively small sections (or no section) have been amplified, the presence and size of the insert will prevent further amplification, effectively removing or reducing the presence of these undesired species from the reaction mixture. This embodiment of the method is generally outlined in FIG. 1D.

Following the step 1150, one can optionally amplify the double-extended loopable primer using an amplification primer 1160. The amplification primer will have a sequence that will hybridize to the complement of the universal region (e.g., it will include a universal region sequence) and, optionally, a sequence that will hybridize to the complement of the noncomplementary region. In some embodiments, one can then allow the double-extended loopable primer to self-hybridize 1170. In some embodiments, the primer-dimers and other short length double-extended loopable primers more readily form the self-hybridized structures than the longer double-extended loopable primers, thereby effectively removing these structures from amplifications. On the other hand, longer double-extended loopable primers can take longer to self-hybridize, giving the amplification primer enough time to anneal and amplify these longer double-extended loopable primers. In some embodiments, the longer double-extended loopable primers are so long that even when self-hybridized, there is sufficient space as to allow amplification of the insert section. Thus, the self-hybridized structure (of at least the shorter insert section containing double-extended loopable primers) can then be used for the selective amplification of large insert sections over relatively small insert sections 1180 (depicted in FIGS. 4 and 5 with respect to linear primers). The use of the loopable primer described above results in a self-hybridized structure that allows for the selective amplification of larger sections of target nucleic acid sequences over smaller sections of target nucleic acid sequences contained within the self-hybridized structures. In some embodiments an initial reverse transcription step can be performed or a cleaning step can be included (or excluded), as described in the following sections.

The following sections describe additional various embodiments. While the figures generally depict linear primers as an example of the “target primer,” one of skill in the art will understand that the descriptions (and relevant portions of the figures) apply equally to the loopable primers described above. Thus, the following embodiments apply and are meant to describe both linear and loopable primer embodiments (unless stated otherwise).

General Target Primer Uses and Embodiments

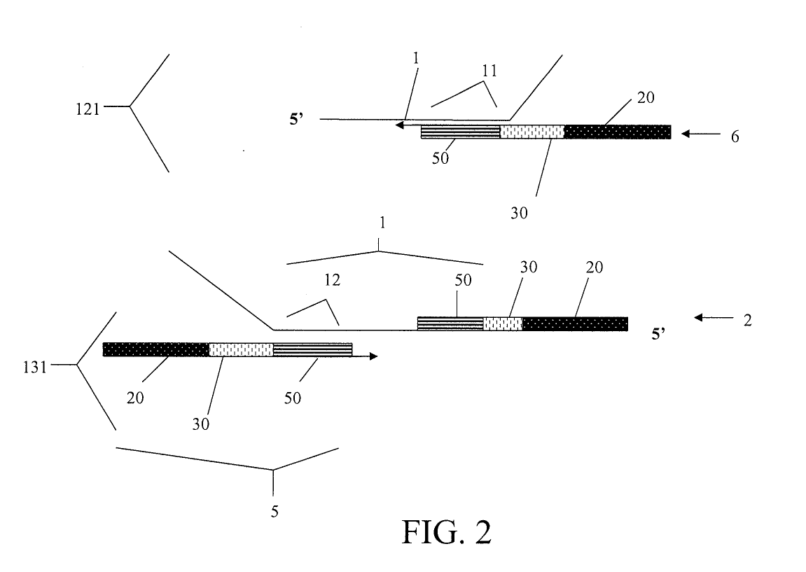

Additional embodiments of the method of using the target primers for the selective amplification of relatively larger target nucleic acid sequences (compared to shorter target nucleic acid sequences) are shown generally in FIG. 1E. The first step 200 can involve primer extension via the target primers described above (to form a double-extended target primer) which can be followed by step 210, a digestion of various random primers, such as with exonuclease I. In some embodiments, this is followed by a pre-PCR amplification step with a single amplification primer (step 220). Following this, a step is performed to amplify the insert section, depending upon the size of the target nucleic acid sequence within the insert section. This can be achieved with an insert amplification primer (step 230). As shown in FIG. 1E by the arrows, various steps can be included or removed for various embodiments. In some embodiments, the cleaning step 225 is not performed or is performed after the pre-PCR amplification 220. In some embodiments, multiple rounds of cleaning (e.g., exonuclease digestion) are employed. Specific embodiments involved in these methods are discussed in more detail in regard to FIGS. 2-7.

In the top section of FIG. 2, the target primer 6 is shown hybridized at a first part 11 at a complementary portion of the target nucleic acid sequence 1 in a first arrangement 121. This results from a first step in which the target primer 6 is allowed to anneal via the 3′ target specific region 50 to the first part of the target nucleic acid sequence at a target binding site 11. Following the hybridization, the primer is extended along the target sequence in the 5′ direction of the target sequence or in the 3′ direction from the target primer (arrow). Following this extension, an additional target primer 5 (which can have the same sequence as the first target primer, a different sequence (but same universal region 20 and/or noncomplementary region 30), and/or the same 3′ target specific region 50 and/or universal region 20 and/or noncomplementary region 30), and/or the same 3′ target specific region 50 and/or universal region 20) hybridizes at a complementary portion of the extended target primer 2 at a second target binding site 12, as shown in FIG. 2, in a second arrangement 131. As above, the target primer 6 can include a 3′ specific target region 50, a noncomplementary region 30, and a universal region 20. In some embodiments, the target primers 5 and 6 are the same. In some embodiments, the target primers are the same, apart from their 3′ target specific region 50. For ease of explanation, the present figures depict embodiments in which target primers 5 & 6 are the same sequence (apart from embodiments in which the 3′ target specific region 50 is degenerate). However, one of skill in the art will readily appreciate how the sequences within these target primers 5 & 6 can be differed, if desired. One of skill in the art will appreciate that various adjustments can be made, as long as the two primers can still hybridize as shown in FIGS. 4 and 5.

In some embodiments, the 3′ target specific region is a degenerate region; thus, identifier “50” can represent multiple or different sequences on different primers as it can be a degenerate sequence. For FIGS. 3-7, the 3′ target specific region is depicted as identifier 50 and 52, (to provide additional clarity for some embodiments in which the 3′ target specific region is degenerate), and thus the specific sequences of 50 and 52 are identified by different identifiers in the figures. However, both 50 and 52 can be a 3′ target known or specific region (and thus can be the same in some embodiments). In addition, the 3′ target specific region identifier “50” can be used generically throughout a single FIGURE (such as in FIG. 2), to denote different sequences, even though a single identifier is used (thus, “50” and “52” need not be present to denote that a region is degenerate). One of skill in the art will readily appreciate how this and other sequences within these linear primers 5 & 6 can be differed, if desired.

Following the hybridization of the target primer 6 to the extended target primer 2 the target primer 6 is extended from its 3′ direction to the 5′ direction of the extended target primer. This extension results in a double-extended target primer 4 (FIG. 3). As noted above, the term “double-extended target primer” does not imply that the sequence functions as a primer, but that it is formed from extending target primers.

The double-extended target primer can optionally be amplified at this point. This is shown in more detail in FIG. 3 in which an amplification primer 60 is used to amplify the double-extended target primer 4. In some embodiments, the amplification primer comprises, consists, or consists essentially of the universal region 20. In some embodiments, the amplification primer includes the noncomplementary region 30 and a universal region 20. This amplification primer 60 can hybridize to the double-extended target primer allowing for efficient amplification of the double-extended target primer. In some embodiments, more than one amplification primer can be used. In some embodiments, only a single primer per target primer nucleic acid sequence is used in the amplification step depicted in FIG. 3. In some embodiments, the use of a single primer sequence that will not hybridize to the initial target primer can help reduce nonspecific primer dimerization that could otherwise occur due to the presence of an amplification primer and remaining target primers. Thus, by selecting an amplification primer that has the same sequence as a portion of the target primer, one can further reduce the risk of primer dimerization or other nondesired hybridization events. Of course, the presence of the noncomplementary region 30 in the target primer 6 can be exploited in selecting such an amplification primer 60. In some embodiments, the amplification of the double extended linear primer results in the selective amplification of double extended linear primers having long insert sections over those with shorter or no insert sections.

As will be appreciated by one of skill in the art, the amplification step can occur in situations in which additional background DNA or nucleic acid sequences are present. As will be appreciated by one of skill in the art, in embodiments in which the linear amplification primer only hybridizes to the universal region, there could be significant priming events to non target sections. However, the presence of the noncomplementary region in the target primer (and more specifically sequences complementary to these regions in the double-extended target primer) and in the amplification primer reduce the likelihood that this will occur.

Following the optional amplification step, at least a subpopulation of the double-extended target primer can self-hybridize (e.g., to achieve the configuration 108, as shown in FIG. 5). As noted above, self-hybridization of the double extended target primer does not have to occur for all species in a sample. Rather, self-hybridization need only occur for the shorter sequences (FIG. 5) which are to be reduced or amplified over. Thus, in some embodiments, self-hybridization occurs for the structures in FIG. 5, but not for the structures depicted in FIG. 4. However, in some embodiments, the longer double-extended target primers also self-hybridize, as shown in FIG. 4.

As will be appreciated by one of skill in the art, the portions of the double-extended target primer corresponding to the universal region 20 and the universal region complement 20′ are capable of hybridizing to one another. The insert section 9 itself can then have the target nucleic acid sequence, or fragment thereof, which can be amplified (for example by PCR). In some embodiments, insert amplification primer(s) 80 and/or 81 are used to amplify at least a portion of the insert. As will be appreciated by one of skill in the art, the size of the insert can be sufficient to allow amplification.

In embodiments in which self-hybridization of the longer double extended target primers is not required to occur (e.g., does not occur frequently or is not driving a subsequent selective amplification of longer insert sections over shorter insert sections), then the selective amplification can occur due to the fact that the shorter double-extended target primers self-hybridize more rapidly than the longer double-extended target primers and thus are removed from subsequent rounds of amplification more quickly than the longer double-extended target primers. In such embodiments, while self-hybridization still occurs for the shorter double-extended target primers (e.g., primer dimers) it does not need to occur for the longer double-extended target primers. As the universal region of the target primer and the universal region complement of the target primer complement on these longer double-extended target primers (as depicted in FIG. 4) are separated by more nucleotides than the shorter double-extended target primer (FIG. 5), the self-hybridization of the longer double-extended target primers will take longer, allowing more time for the insert amplification primer to hybridize and extend. Thus, the self-hybridized structure for the longer double-extended target primer need not be formed to selectively amplify the longer double-extended target primer over the shorter double-extended target primer.

As will be appreciated by one of skill in the art, in embodiments in which whole genome amplification is being performed, the precise sequence within the insert section can be unknown. In light of this, it can be advantageous to use multiple insert amplification primers to make certain that one will prime and extend as desired. In some embodiments, a pool of insert amplification primers is used. In other embodiments, one insert amplification primer (and/or one set or more) is mixed with the solution containing the double-extended target primer. As will be appreciated by one of skill in the art, numerous such mixtures (e.g., 2-10, 10-100, 100-1,000, 1,000-10,000 or more) can be done in series or in parallel. Furthermore, the solution containing the double-extended target primer can be divided into parts so that the various reactions can be run in parallel.

As will be appreciated by one of skill in the art, not every target primer will necessarily hybridize to the target sequence as desired and in some embodiments a target primer duplex or primer dimer will be formed. For example, in some situations, subsequent primers (such as an amplification primer) can hybridize to the target primer complement, resulting in only the amplification of the primer. Additionally, in some embodiments, target primers can hybridize to one another, also forming short amplification products. Additionally, in some embodiments, nonspecific hybridization or overly frequent hybridization of the 3′ target specific region or of other sections (such as the universal region) of the various primers to sections of the target nucleic acid sequence can occur such that only these smaller sections of the target nucleic acid sequence are amplified. One depiction of the above is shown in FIG. 5. In such a situation, rather than having target nucleic acid sequence (or a significant amount of it) between the universal region 20 and the complement to the universal region 20′, there is an insignificant amount of target sequence between the two 20 and 20′. As shown in FIG. 5, when the universal region 20 and universal region complement 20′ hybridize together under this situation, the insert section 109 in the complex 108 is relatively small. In some embodiments, there is a nucleic acid sequence 51 in the insert section between the 3′ target specific region 50 and 52. This nucleic acid sequence 51 need not be present and, if it is present, is relatively short. In some embodiments, (when a sufficiently large insert is present) the insert 9 (including sequence 51) is not more than 10 kb in length. In some embodiments, the insert 109, while still capable of allowing amplification does so with relatively less efficiency than the double-extended target primer complex 8 shown in FIG. 4 (which, of course, need not actually assume the structure shown during the process). As such, relative amplification of the product 8 (or 4 in the non-self-hybridized form) shown in FIG. 4 can be achieved compared to amplification of the resulting product 108 shown in FIG. 5. As will be appreciated by one of skill in the art, this distinction between the two resulting products can reduce the role or impact that nonspecific primer interactions can have. That is, this distinction can generally improve target detection or sequence amplification by reducing the impact of nucleic acid structures (or products) in which a significant or substantial amount of target DNA has not incorporated between the two primers. As will be appreciated by one of skill in the art, when the 3′ target specific region 50 and 52 are complementary to one another (e.g., when only a single sequence is used and the 3′ target specific region is not a degenerate sequence) the complement 52 can be hybridized together and the sequence 51 need not be present (e.g., when the double-extended target primer is just a primer dimer). In embodiments in which the 3′ target specific region is a degenerate region or sequence, then sections 50 and 52 need not, and often will not, be complementary to one another.

While not depicted in FIGS. 4 and 5, one of skill in the art will readily recognize that in the embodiments in which a self-hybridized structure is not created for the longer double-extended linear primer, that the insert amplification primers 81 and 80 can bind to the “open” double-extended linear primer, and can bind to the universal region or other section of the linear primer. In some embodiments, one of the insert amplification primers comprises, consists, or consists essentially of a universal region, while the second insert amplification primer primes in the insert. In some embodiments, both insert amplification primers hybridize within the insert (as shown in FIG. 4, although no actual loop need be formed). In some embodiments, neither of the insert amplification primers prime or overlap with any section of the linear primer.

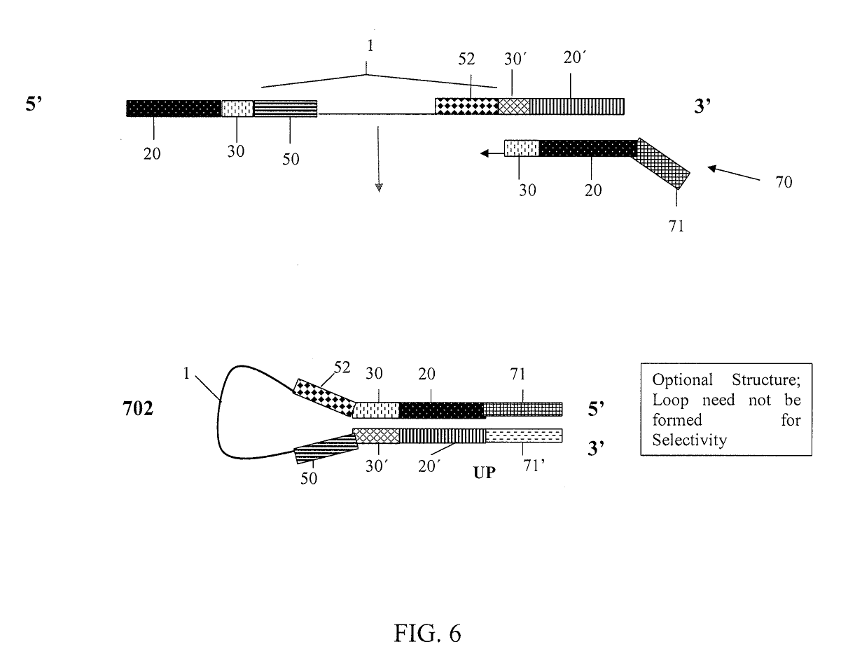

As will be appreciated by one of skill in the art, in some embodiments, it is desirable to have specific sequences on the 5′ and/or 3′ end of the nucleic acid sequence that have been amplified, such as the double-extended target primer. Examples of such specific sequences include zip-code sequences, as described in U.S. Pat. Pub. No: 2006/0014191 (the entirety of which is hereby incorporated by reference). One option for achieving this is shown in FIG. 6 and FIG. 7 (which depict the self-hybridized embodiments only, although one of skill in the art can adjust the figures for the non-self-hybridized embodiments as well). In such embodiments, rather than (or following) the amplification step depicted in FIG. 3 involving the amplification primer 60 (which can comprise, consist, or consist essentially of a universal region), one performs an amplification step to add a desired sequence (e.g., 71) to one end of the double-extended target primer via a different primer 70. This process, and the resulting product 702 are shown in FIG. 6 for a double-extended target primer that has a significant amount of target nucleic acid sequence in it, and in FIG. 7 (802), for a double-extended target primer that has an insignificant amount of target DNA in it.

In some embodiments, there is a first amplification primer 70 which, while including the universal region 20 (and optionally the noncomplementary region), includes an additional section 71. This section 71 allows one to customize the end(s) of the double-extended target primer. As will be appreciated by one of skill in the art, section 71 is not a “noncomplementary” region, as defined herein, rather, it is a sequence that is not complementary to the sequence that the amplification primer 70 is hybridized to. The ability to have different sequences on each end of the nucleic acid segment can be useful in some sequencing applications. Thus, the above amplification primer 70 can be used in these situations. The primer 70 can include the noncomplementary region 30 and the universal region 20. As will be appreciated by one of skill in the art, different primers 70, each having a different section 71, can be added to specific double extended linear primers, allowing various double extended linear primers to be combined. In some embodiments, more than one amplification primer is used (e.g., two or more different sequenced primers, as depicted in FIG. 6 and processed in parallel, while still being able to identify the specific double extended linear primer). Of course, this can be adjusted for loopable primers as well.

As shown in the lower section of FIG. 6, when the target nucleic acid sequence 1 is included, amplification proceeds from these two primers to produce a double-extended target primers 702 (which need not be self-hybridized).

In contrast, as shown in FIG. 7, in those situations in which very little or no target nucleic acid sequence is included between the universal region 20 and its complement 20′, the resulting structure has a relatively smaller insert section resulting in relatively less amplification through the use of the insert amplification primers (802) and/or the optional amplification step depicted in FIG. 3 (as noted above, this can be due to the faster hybridization kinetics due to the shorter linker and/or due to the smaller size of the loop structure which can physically limit processing of this area.

### MDA Techniques

While isothermal multiple displacement amplification (“MDA”) can be used for genome wide amplification, the techniques for employing this method have previously been constrained in various ways in order to produce useful amplification results.

It has presently been appreciated that employing a herein disclosed target primer in a MDA process can produce specific advantageous results. Furthermore, it has been appreciated that using a herein disclosed target primer in a hybrid MDA and PCR technique can further provide various advantages over the techniques that are presently available for whole genome amplification. In some embodiments, the use of a target primer to add a universal region and a complement of a universal region to two ends of a target sequence, and thereby create a self-selecting amplification process, allows for the effective silencing (e.g., reduction) of the shorter products that could dominate standard MDA and/or PCR amplification processes.

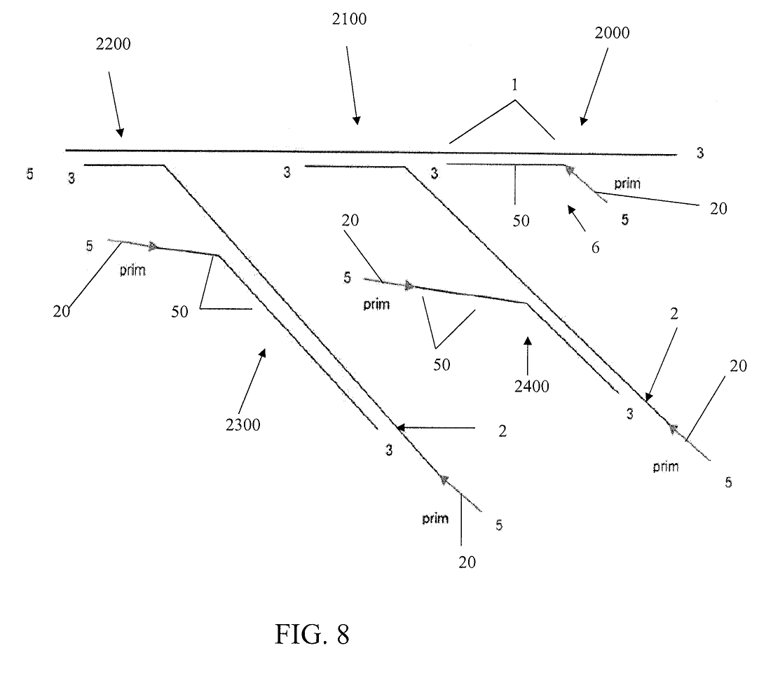

An embodiment of the above process is generally depicted in FIG. 8, which generally outlines an MDA process employing a target primer. As can be seen in the figure, during the MDA process, multiple priming and extension events are occurring on the target nucleic acid sequence. For example, as shown in FIG. 8, there can be a target primer 6 hybridized to a target nucleic acid sequence 1, as shown on the right-hand side at location 2000, as well as nucleic acid sequences that are being extended (and displaced) from the target primer (and are in the 5′ direction from the target nucleic acid sequence 1 (such as shown at locations 2100 and 2200)). Thus, there can be multiple extended target primers 2, still hybridized 5′ to the initial target sequence 1.

Furthermore, in some embodiments, further amplification (e.g., MDA and/or PCR) can occur by priming off of these initial extended target primers 2. As shown in FIG. 8, in some embodiments, another target primer, which can include the same or a different 3′ target specific region 50 (and can include the same universal region as it is a target primer), can hybridize to the extended target primers 2. This in turn can, if allowed to extend through the end of the extended target primer 2, form a double extended target primer 4 (as shown in FIG. 4). FIG. 8 depicts at least two separate extended target primers 2300 and 2400 going through this extension and coming close to becoming double extended target primers. In some embodiments, the further amplification is a PCR amplification. In some embodiments, this PCR amplification occurs before the formation of a significant amount of a hyper-branched product, in the MDA reaction. In some embodiments, the MDA reaction is stopped prior to the formation of a hyper-branched product, or a significant amount of a hyper-branched product, in the MDA reaction. In some embodiments, this second round of amplification is a MDA reaction. In some embodiments, the MDA reaction is stopped before at least 1% of the product from the MDA reaction is hyper-branched, for example, at least 1, 2, 3, 4, 5, 10, 15, 20, 25, 30, 35, 40, 45, 50, 55, 60, 65, 70, 75, 80, 85, 90, 95, 96, 97, 98, 99 percent, or more of the product is a hyper branched product. As will be appreciated by one of skill in the art, a “significant amount” of a hyper-branched product can vary based upon the specific application. The term denotes that the amount of the hyper-branched product formed should not prevent the formation of a double-extended primer. In some embodiments a significant amount is an amount of at least one of the following: 1, 2, 3, 4, 5, 10, 15, 20, 25, 30, 35, 40, 45, 50, 55, 60, 65, 70, 75, 80, 85, 90, 95, 96, 97, 98, 99, or 100 percent.

The embodiment in FIG. 8 depicts a single target sequence 1; however, multiple target sequences can be targeted and amplified with this process, allowing for genome wide amplification. In addition, there can also be multiple nucleic acid sequences that are being extended from the extended-target primers to form the double-extended target primers. As will be appreciated by one of skill in the art, once the double-extended target primer is created, it can have the ability to self-hybridize to some extent (depending, of course, upon temperature of the mixture, other nucleic acids sequences available, the amount of time, and other variables appreciated by those of skill in the art); thus, the self-hybridization of the shorter double-extended target primers can begin to influence the product at this point and, in some embodiments, this selective ability can continue from that point through to the use of the insert primers.

As noted above, in some embodiments, the nucleic acid sequences from the isothermal amplification can display the self-hybridization properties noted above; thus, in some embodiments, the initial selection of longer insert sections over shorter insert section (such as with primer dimers) can begin at this point.

In some embodiments, following (or during) either the creation of the extended target primer or the creation of the double extended target primer, a PCR amplification can be performed using a target primer that will have the same universal region as the initial target primer used in the MDA step. In some embodiments, this target primer can be degenerate (and thus include multiple primers, with different 3′ target specific regions). As noted herein, there are additional advantages for combining both MDA and PCR when using the herein disclosed target primers for genome wide amplification.

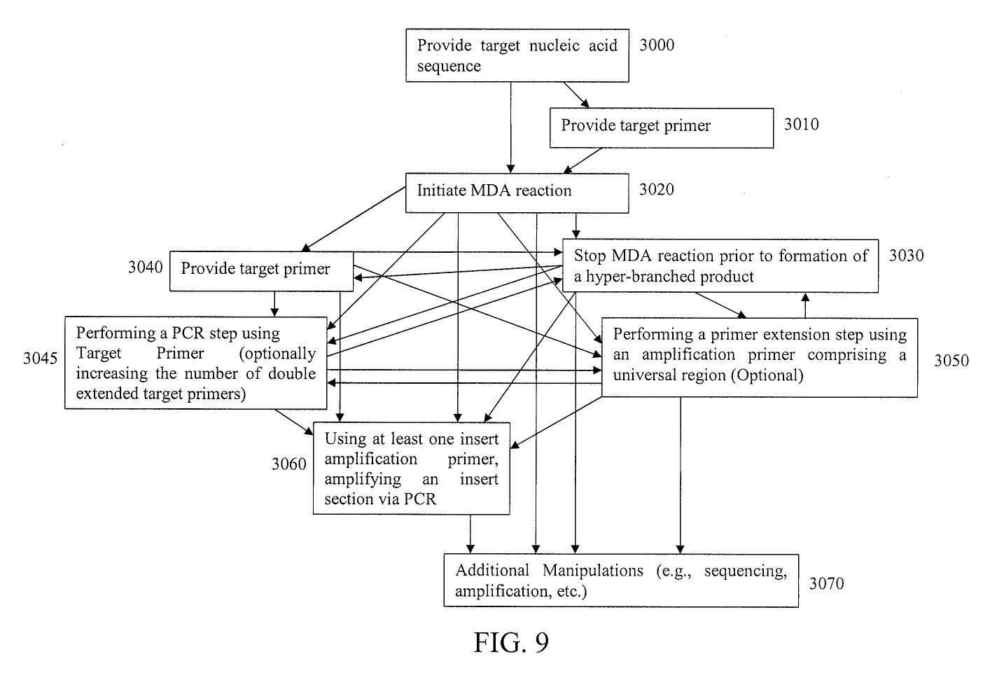

While there are numerous embodiments by which the above MDA or hybrid MDA/PCR process can occur, some of these embodiments are generally depicted in FIG. 9, which shows various pathways of making a double extended target primer and/or using a target primer.

In some embodiments, the method involves providing a target nucleic acid sequence 3000, providing a target primer 3010 (which will at least comprise a universal region and a 3′ target specific region and which can be a MDA operable primer), initiating a MDA reaction 3020, stopping or attenuating the MDA reaction prior to the formation of a significant amount of a hyper-branched product, 3030, performing a PCR amplification using a target primer 3045 (the target primer need not be a MDA operable primer), using an amplification primer that comprises, consists, or consists essentially of a universal region to amplify from the complement of the universal region on the double extended target primer 3050, and using at least one insert amplification primer (and in some embodiments adding at least two insert amplification primers) to amplify an insert section 3060, which can be done through PCR.

Other embodiments depicted in FIG. 9 include, for example, proceeding through processes 3000, 3010, 3020, 3040, 3045, 3030, and 3060; processes 3020, 3040, 3030, 3045, and 3060; processes 3000, 3010, 3020, 3030, 3040, 3045, 3050, 3060, and 3070; processes 3020, 3030, 3040, 3045, and 3060; processes 3020, 3030, 3045, and 3060; processes 3000, 3010, 3020, and 3060; processes 3020 and 3060; and processes 3000, 3010, 3020, 3030, 3040, 3045, 3050, 3060, 3070. Of course, even those pathways depicted in FIG. 9 that are not explicitly listed above are also alternative embodiments. One of skill in the art will appreciated that the various pathways depicted in FIG. 9 can, in some embodiments, include additional processes, and in other embodiments, exclude any significant additional processes. Furthermore, in some embodiments, the entire method can start at process 3020 and continue from there. In some embodiments, additional options can be included prior to process 3000, or after process 3070. As will be appreciated by one of skill in the art, unless explicitly noted, the various processes depicted in FIG. 9, need not stop as one progresses through a pathway. Thus, in some embodiments, a method or pathway that proceeds from process 3020 to process 3040 to process 3045, can still have some MDA reaction occurring even during process 3045. Moreover, as noted herein, in some embodiments, process 3030, while attenuating the MDA process, does not have to stop 100% of the MDA process (although it can result in this in some embodiments).

In some embodiments, the processes noted in FIG. 9 occur in the order in which they are depicted in FIG. 9, from top to bottom, following the arrows. In some embodiments, one or more of the process is stopped (or attenuated) before or as the next step is started. In some embodiments, the double extend target primer is formed at any one of processes 3000, 3010, 3020, 3030, 3040, 3050, or 3060. In some embodiments, the double extended target primer is formed during at least one of the following processes: 3000, 3010, 3020, 3030, 3040, 3050, or 3060. In some embodiments, the double extended target primer is formed at process 3020 and onward. In some embodiments, the double extended target primer is formed at process 3030 and onward. In some embodiments, the double extended target primer is formed at process 3040 and onward. In some embodiments, the double extended target primer is formed at process 3045 and onward. In some embodiments, the double extended target primer is formed at process 3050 and onward. In some embodiments, a self-hybridized structure for a double extended target primer, that is a primer dimer, is created at process 3020 and/or onward. In some embodiments, a self-hybridized structure for a double extended target primer, that is a primer dimer, is created at process 3030 and/or onward. In some embodiments, a self-hybridized structure for a double extended target primer, that is a primer dimer, is created at process 3040 and/or onward. In some embodiments, a self-hybridized structure for a double extended target primer, that is a primer dimer, is created at process 3050 and/or onward. In some embodiments, a self-hybridized structure for a double extended target primer, that is a primer dimer, is created at process 3060 and/or onward.

In some embodiments, following the creation of the double extended target primer, a pre-PCR amplification step can occur, such as that depicted in FIG. 3, to further amplify the double extended target primer. The pre-PCR amplification can employ the amplification primer 60, which will at least have the universal region 20. As noted above, this pre-PCR amplification can also have the self-hybridization properties noted above; and thus, the initial selection of longer insert sections over shorter insert section (such as with primer dimers) can begin at this point.

In some embodiments, the MDA reaction occurs in the presence of a PCR amplification enzyme.

As noted herein, the insert amplification can be done without actually forming a self-hybridized structure for the longer double extended target primers. In some embodiments, only the double-extended target primers that are primer dimers are self-hybridized during the insert amplification process 3060. In some embodiments, at least the double-extended target primers that are primer dimers are self-hybridized during the insert amplification process 3060. In some embodiments, the double-extended target primers that are self-hybridized during the insert amplification process 3060 comprise an insert section less than 200 nucleotides in length. In some embodiments, the double-extended target primers that are self-hybridized during the insert amplification process 3060 comprise an insert section less than 100 nucleotides in length. In some embodiments, at least the double-extended target primers that comprise an insert section less than 100 nucleotides in length are self-hybridized during the insert amplification process 3060. In some embodiments, at least the double-extended target primers that comprise an insert section less than 200 nucleotides in length are self-hybridized during the insert amplification process 3060.

As will be appreciated by one of skill in the art, as long as a self-hybridized or hybridizable structure is formed at some point in the process (for at least the shorter products, such as a primer dimer), at least some of the advantages disclosed herein can be achieved. Thus, in some embodiments, the universal region and the universal region complement are placed in one strand having an insert section between the two (thus a double extended target primer is formed), prior to the MDA process, during the MDA process, following the MDA process, prior to the formation of a significant amount of a hyper-branched product, before the pre-PCR step, prior to the PCR step, during both the MDA and PCR steps, during the pre-PCR step, during the PCR step, and/or following the PCR step.

In some embodiments, both MDA and PCR can occur at the same time, during overlapping time periods, or sequentially and separately. In some embodiments, the MDA process is stopped (e.g., attenuated) before PCR is performed.

As will be appreciated by one of skill in the art, any of the above processes (or those noted in FIG. 9) need not be 100% stopped (or, in some embodiments, stopped at all) before proceeding to a next or subsequent step. In embodiments in which one process is “stopped” prior to proceeding to the next step, such a stopping need only be adequate to allow the process to achieve an end goal (e.g., selective amplification of insert DNA over shorter insert sections, such as primer dimers). Thus, in some embodiments, one or more earlier step(s) can continue to occur through later steps. In some embodiments, when a reaction is “stopped,” a significant portion of the reaction only needs to be stopped. Thus, in some embodiments, at least 10, 10-20, 20-30, 30-40, 40-50, 50-60, 60-70, 70-80, 80-85, 85-90, 90-95, 95-98, 98-99, 99, 99-100 percent of the reaction is stopped. This can be measured in regard to the amount of product from the reaction that continues to be made, with a 100% stopping of the reaction resulting in 0 percent of the product being made. This can also be referred to as “attenuating” the reaction, step, or process. Thus, in the present disclosure, “stop” only requires an adequate attenuation of the process. If a complete halt to a process is intended, it can be denoted as a “complete stop” or “stopping 100%” of a process. Of course, even at this level of stopping, there can often be individual molecules that may still function.

As noted herein, in some embodiments, the MDA reaction is stopped or attenuated prior to a formation of a hyper-branched product, or a significant amount of a hyper-branched product, in MDA amplification. In some embodiments, some of the MDA reactions can proceed to a hyper-branched product in the MDA reaction, as long as at least one MDA reaction (involving at least one nucleic acid strand) is stopped prior to the formation of a hyper-branched product in the MDA amplification. In some embodiments, at least one nucleic acid strand in a solution is stopped prior to the formation of a hyper-branched product in the MDA reaction. In some embodiments, at least 0.001% of the MDA reactions present in the mixture are stopped prior to the formation of a hyper-branded product of the MDA reaction, for example, 0.001, 0.01, 0.1, 1, 1, 5, 10, 20, 30, 40, 50, 60, 70, 80, 90, 95, 98, 99, or 99.9% of the reactions (or any amount above any of the identified values or any range defined between any two of the identified values) are stopped prior to resulting in a hyper-branched product. In some embodiments, the MDA reaction is stopped (or a step is performed to stop the MDA reaction) before or at mid-log. In some embodiments, the MDA reaction is stopped (or a step is performed to stop the MDA reaction) just after mid-log.

In some embodiments, the MDA process is stopped (e.g., attenuated) by commencing the PCR reaction. In some embodiments, a change in temperature from the PCR reaction stops the MDA reaction. In some embodiments, the MDA process is stopped independently from any other process. In some embodiments, the MDA process is stopped or attenuated by elevating the temperature of the reaction solution. Any elevation that can adequately attenuate the MDA process can be adequate. In some embodiments, the temperature is above 50° C., for example 50, 55, 60, 61, 62, 63, 64, 65, 70, 75° C., or higher. This temperature need only be held long enough to provide an adequate level of attenuation of the MDA process. In some embodiments, the temperature is held for at least 1, 2, 3, 4, 5, 6, 7, 8, 9, 10, 20, 30, 40, 50, 60, or more minutes. In some embodiments, the addition of a sufficient amount of PCR enzyme effectively stops or out competes the MDA reaction. In some embodiments, the MDA reaction is attenuated or stopped by changing the buffer of the MDA reaction to a PCR reaction buffer. In some embodiments, the MDA reaction is stopped by filtering out or removing the MDA enzyme or by removing the DNA. In some embodiments, the MDA reaction is stopped by adding a blocker to the MDA enzyme, e.g., an antibody. In some embodiments, the MDA reaction is stopped by degrading or denaturing the MDA enzyme. In some embodiments, the MDA reaction is stopped and then the PCR or pre-PCR step is commenced.

In some embodiments, the target primer for the MDA reaction (“MDA primer”) differs from the target primer for the PCR reaction (“PCR primer”). In some embodiments, the primer(s) are functionally identical (for example they can include a same universal region and include a random and/or degenerate region, and thus that specific sections can differ) apart from the presence of at least one 3′ phosphothioate bond in the MDA primer. While the 3′ regions of such primers need not be identical, the remaining sections of the primers can be. In some embodiments, the MDA and PCR primers will at least have the same universal region. In some embodiments, the MDA primer comprises more than one 3′ phosphothioate bond, such as two or three such bonds. In some embodiments, the PCR target primers lack such bonds. In some embodiments, the PCR primers also include such a bond. In some embodiments, the primers for the PCR and MDA processes are completely identical, including the 3′ target specific region (thus are not random and/or degenerate, unless all possible random and/or degenerate sequences are present for both the MDA and PCR primers).

In some embodiments where the MDA primers are different from the PCR primers, the methods or kits associated with various embodiments disclosed herein can include a primer or primer set that is appropriate for MDA reactions and a primer or primer set that is appropriate for PCR amplifications. In some embodiments, the MDA primers comprise some aspect that can attenuate exonuclease 3 digestion (such as at least one 3′ phosphothioate bond at the 3′ end). In some embodiments, the MDA target primer(s) comprise at least 2 3′ phosphothioate bonds at the 3′ end of the primer. In some embodiments, methods or kits that involve a MDA event can include such primers (or other primers that are resistant to exonuclease digestion).

In some embodiments, primers can be added throughout the method. In some embodiments, a target primer is added after the MDA process and before the PCR process. In some embodiments, an additional amount of a previously added primer can be added. In some embodiments, a different target primer is added at a later step. In some embodiments, all of the primers in the method (at least before the insert amplification process) include a universal region. In some embodiments, all of the primers in the processes noted in FIG. 9 include a universal region (at least before the insert amplification process). In some embodiments, all of the primers used prior to process 3060 in FIG. 9 comprise a universal region. In some embodiments, all of the primers between process 3000 and before process 3060 include a universal region. In some embodiments, the primers involved in processes 3010, 3040, 3045, 3060, and 3050 include a universal region. In some embodiments, the primers involved in processes 3010, 3040, and 3050 prime via a universal region. In some embodiments, the primers employed in process 3060 do not include a universal region. In some embodiments, a single primer is added to perform process 3060. In some embodiments, at least two primers are added to perform process 3060, and these primers bind to an insert section in the double extended target primer.

In some embodiments, the enzyme employed for the initial amplification process (e.g., 3020) is a MDA enzyme or any enzyme that is highly processive. In some embodiments, the enzyme is selected from the group consisting of: Φ29, BST, and any highly processive polymerase.

In some embodiments, the temperature of the MDA reaction is between 5 and 50 degrees C.

In some embodiments, the technique employs the benefits of MDA, for example, retaining relatively even gene amplification.

In some embodiments, the technique avoids primer-related background present in MDA. In some embodiments, one or more of the limitations on traditional MDA whole genome amplification can be removed.

In some embodiments, the technique avoids (e.g., reduces) jackpot mutation effect of PCR on low or single copy molecules because the PCR is done at a multicopy level. In some embodiments, the technique can be used to avoid (e.g., reduce) nonspecific (primer-based) background amplification. In some embodiments, the method employs the advantages of both MDA and PCR based amplification for a method that is useful in low level DNA amplification (e.g., single cell levels, e.g., pictogram levels).

In some embodiments, the process can produce whole genome amplification DNA that can be readily reamplified via standard PCR.

In some embodiments, the MDA reaction is performed as described in one of the following references: “Cell-free cloning using phi29 DNA polymerase,” Hutchison III et al., (PNAS, 102:17332-17336, 2005); “Genome coverage and sequence fidelity of phi29 polymerase-base multiple strand displacement whole genome amplification,” Paez et al. (Nuc. Acids Res. Vol. 32 No. 9, e71, 2004); “Genomic DNA Amplification from a Single Bacterium, Raghunathan et al.,” (Applied and Environmental Microbiology, Vol. 71, No. 6, 3342-3347, 2002); “Comprehensive human genome amplification using multiple displacement amplification,” Dean et al., (PNAS, Vol. 99, No. 8, 5261-5266, 2002); and Sequencing genomes from single cells by polymerase cloning, Zhang et al., (Nature Biotech. Vol 24, No. 6, 680-686, 2006), the entireties of each of which is herein incorporated by reference.

In some embodiments, the MDA reaction parameters and/or ingredients can be as follows: dNTP, target primer (with two phosphothioate bonds) RepliPHI reaction buffer, at 30° C. for 10 hours; 37 mM TrisHCL (pH7.5), 50 mM KCl, 10 mM MgCl₂, 5 mM (NH₄)₂SO₄, 1 mM dNTPs, 50 micromolar exonuclease resistant primer, pyrophosphatase for 18 hours at 30° C., stopped by heating to 65° C. for 3 minutes; a REPLI-g™ 625S amplification kit, (Molecular Staging Inc., New Haven Conn.); and/or 37 mM TrisHCL (pH7.5), 50 mM KCl, 10 mM MgCl₂, 5 mM (NH₄)₂SO₄, 1 mM dNTPs, 1 mM DTTXBSA, 0.2% Tween 20, 1 unit/ml yeast pyrophosphatase, and exonuclease resistant primer. Of course, these are merely exemplary conditions and one of skill in the art will appreciate how they can be adjusted for particular situations.

In some embodiments, MDA is replaced by a different or related technique that involves a highly processive enzyme. In some embodiments, the technique employed is rolling-circle amplification and/or ramification amplification, as described in Yi et al., Nuc. Acids. Res. Vol. 34, No. 11, e81, entitled: “Molecular Zipper: a fluorescent probe for real-time isothermal DNA amplification” (incorporated herein in its entirety by reference). Thus, in some embodiments, the RAM reaction (its primers and reaction parameters) described in Yi et al. can be used in place of the MDA reaction described herein.

In some embodiments, the 3′ target specific region is the same for each 3′ target specific region in the target primer. In some embodiments, the 3′ target specific region is different. In some embodiments, the 3′ target specific region can comprise a degenerate sequence or random region, and thus, the primer comprises numerous different primers, at least some of which have different sequences at the 3′ target specific region.

In some embodiments, the target nucleic acid sequence is derived from a whole genome. In some embodiments, the target nucleic acid sequence is from a single cell. In some embodiments, the target nucleic acid sequence comprises genomic DNA.

In some embodiments, the target primer is a linear primer when it hybridizes to the target nucleic acid sequence. In some embodiments, the first target primer is a looped primer when it hybridizes to the target nucleic acid sequence.

In some embodiments, only a single universal region primer sequence is used in the PCR amplification (and/or pre-PCR) of any given PCR (and/or pre-PCR) amplified nucleic acid sequence. In some embodiments, in the PCR (and/or pre-PCR) amplification, only a single PCR (and/or pre-PCR) primer is used to amplify all of the PCR (and/or pre-PCR) amplified nucleic acid sequences. In some embodiments, in the PCR (and/or pre-PCR) amplification, only a single universal region nucleic acid sequence is used as a primer to amplify all of the PCR (and/or pre-PCR) amplified nucleic acid sequences.

In some embodiments, the temperature of the solution containing the double extended target primers is cooled, thereby allowing at least the shorter of the double-extended target primers to self-hybridize via the universal region and the sequence that is complementary to the universal region.

In some embodiments, the above technique is applied in whole genome amplification (WGA). In some embodiments, the technique avoids or reduces the impact of priming between random primers in techniques such as primer extension preamplification (PEP) or degenerate oligonucleotide primed PCR (DOP-PCR). In some embodiments, when utilizing the aspects disclosed herein, the random primers need not be limited to specific random regions (for example the random regions can include T and/or C and/or A and/or G), need not be limited to ultra small volumes (e.g., 600 nl or less or 60 nl or less), and/or can be used on subnanogram quantities of starting sample. In some embodiments, one or more of these advantages or aspects are present in the method. In some embodiments, one or more of these aspects can be combined in a method or a kit. Is some embodiments, the amplification is not for whole genome amplification.

In some embodiments, the use of a target primer as described above allows one to analyze especially low amounts of target nucleic acid in whole genome amplification. For example, in some embodiments, the initial sample contains less than 1 gram of target nucleic acid sequence, for example, 1000-100, 100-10, 10-1, 1-0.1, 0.1-0.01, 0.01-0.001, 0.001-0.0001, 0.0001-0.00001, 0.00001-0.000001 nanograms or less. In some embodiments, the amount of target nucleic acid is the amount of the target nucleic acid in a single cell. In some embodiments, the amount of target nucleic acid is between 0.5 and 100 pg. In some embodiments, the amount of target nucleic acid is less than 100 pg. As will be appreciated by one of skill in the art, this can be especially advantageous in whole genome amplification and sequencing.

In some embodiments, any of the methods can be applied in or for a clinical and/or forensics environment. In some embodiments, the technique is applied in molecular oncology. In some embodiments, the technique is applied to a sample that comprises at least one cell, for example 1, 1-10, 10-100, 100-1000, or more cells. As will be appreciated by one of skill in the art, this can be especially advantageous for whole genome amplification and sequencing. In some embodiments, this can be used in laser captured single cells.

In some embodiments, the relatively large increases in amplification are achieved while still maintaining a significant amount of dose response during the amplification. For example, in some embodiments, relatively small amounts of one species to be amplified will still be a relatively small percent of the amplified product (although it could have been amplified, e.g., 100-1,000,000 times). As will be appreciated by one of skill in the art, this can be especially advantageous in whole genome amplification and sequencing.

As will be appreciated by one of skill in the art, while the single primer amplification embodiment ameliorates the problem of random background sequence amplification, it can introduce kinetic parameters that impact the levels of amplification. During the formation of the double extended target primer, with every thermal cycle a significant amount of primer can be removed by primer-primer hybridization and extension. As such, in some embodiments, it can be advantageous to use relatively high levels of primer. In some embodiments, 10 micromolar or more can be used, for example, 10-100, 100-1000, 1000-10,000, 10,000-100,000 micromolar can be used. In some embodiments new primer can be added during or throughout the procedure.

In some embodiments, the target primer (and its methods of use) allows one to use a 3′ target specific region that is not constrained to just A or G in whole genome amplification. In some embodiments, the 3′ target specific region is or includes a random and/or degenerate region. In some embodiments, this region includes T and/or C in the random region. As will be appreciated by one of skill in the art, this does not mean that the region is no longer “random.” In some embodiments, the random region can include at least three different nucleotides (e.g., A, G and T or C; or T, C, and A or G). In some embodiments the random region can include at least four different nucleotides.

In some embodiments, the random region includes at least one thymine. In some embodiments, the random region includes at least one cytosine. In some embodiments, at least one of the primers in the amplification reaction includes a cytosine and/or thymine in the random region. In some embodiments, at least one of the primers in the amplification reaction includes, in the random region, at least one nucleotide that is not an adenine or a guanine. In some embodiments, the base or nucleotide is or comprises a thymine, cytosine, or uracil, nucleotide analog (e.g., including thymine, uracil, and/or cytosine analogs), or other option. In some embodiments, the random region includes at least 2, 3, 4, 5, 6, 7, 8, 9, 10, 11, 12, or more nucleotides that are cytosine and/or thymine. In some embodiments, the random region includes at least 2, 3, 4, 5, 6, 7, 8, 9, 10, 11, 12, or more nucleotides that are not adenine and/or guanine. As will be appreciated by one of skill in the art, removing this constraint can be especially advantageous in WGA applications and sequencing.

In some embodiments, the use of a target primer allows one to analyze samples that are relatively large in volume compared to standard whole genome amplification techniques. For example, in some embodiments, the sample is more than 60 nl, for example, 60-80, 80-100, 100-200, 200-500, 500-600, 600-601, 601, 500-1000, 1000-10,000, 10,000-100,000, 100,000-1,000,000 nl or more in volume. In some embodiments, an initial sample is diluted or brought up to a volume that is above 60 nl, for example, 60-80, 80-100, 100-200, 200-500, 500-1000, 1000-10,000, 10,000-100,000, 100,000-1,000,000 nl or more. In some embodiments, a sample to be analyzed starts off as a dry or non-liquid sample and a volume of liquid is added to the sample to suspend the sample. In some embodiments, the volume used to suspend the sample is more than 60 nl, for example, 60-80, 80-100, 100-200, 200-500, 500-1000, 1000-10,000, 10,000-100,000, 100,000-1,000,000 nl or more. In some embodiments, any one or more of the processes outlined in FIG. 9 is carried out in a volume that is above 60 nl, for example, 60-80, 80-100, 100-200, 200-500, 500-600, 601, 600-1000, 1000-10,000, 10,000-100,000, 100,000-1,000,000 nl or more. In some embodiments, at least one of the amplification processes in FIG. 9 is carried out in a volume that is above 60 nl, for example, 60-80, 80-100, 100-200, 200-500, 600, 601, 500-1000, 1000-10,000, 10,000-100,000, 100,000-1,000,000 nl or more. As will be appreciated by one of skill in the art, removing the volume constraint can be especially advantageous in WGA applications and sequencing.

As will be appreciated by one of skill in the art, the above embodiments can be achieved via the use of a target primer that results in the formation of a double extended target primer that can self-hybridize (at least for the shorter double-extended target primers). Thus, in some embodiments, eventually the amplified DNA will have two sections that can self-hybridize. In some embodiments, this is achieved via the use of a single target primer in the amplification reaction (such that the amplified DNA has a sequence that is the target primer on one end and the complement of the target primer on the opposite end). In some embodiments, this can be achieved via the use of different primers, where all of the primers share a common sequence (such as the universal region, a random region, and/or a noncomplementary region) such that they can still produce the double extended target primer that can self-hybridize.

### Additional Embodiments

In some embodiments, the use of a target primer as described above allows one to analyze especially low amounts of target nucleic acid in whole genome amplification. For example, in some embodiments, the initial sample contains less than 1 gram of target nucleic acid sequence, for example, 1000-100, 100-10, 10-1, 1-0.1, 0.1-0.01, 0.01-0.001, 0.001-0.0001 nanograms or less. In some embodiments, the amount of target nucleic acid is the amount of the target nucleic acid in a single cell. In some embodiments, the amount of target nucleic acid is between 0.5 and 100 pg. In some embodiments, the amount of target nucleic acid is less than 100 pg. As will be appreciated by one of skill in the art, this can be especially advantageous in whole genome amplification and sequencing.

In some embodiments, because the target primers are not biased in how they initially bind to the target nucleic acid sequence (e.g., in contrast to looped primers), they can bind along and within stretches of DNA, thereby avoiding having to over process the gDNA to make relatively short pieces of gDNA for amplification. In some embodiments, the method avoids or does not require overprocessing the initial sample.

In some embodiments, by using the herein presented techniques, one can avoid a precleaning step, such as fragment size selection. Thus, in some embodiments, the method does not include a precleaning step, such as fragment size selection.

In some embodiments, any of the methods can be applied in or for a clinical and/or forensics environment. In some embodiments, the technique is applied in molecular oncology.

In some embodiments, the relatively large increases in amplification are achieved while still maintaining a significant amount of dose response during the amplification. For example, in some embodiments, relatively small amounts of one species to be amplified will still be a relatively small percent of the amplified product (although it could have been amplified, e.g., 100-1,000,000 times).

As will be appreciated by one of skill in the art, while a single primer amplification embodiment ameliorates the problem of random background sequence amplification, it can introduce kinetic parameters that impact the levels of amplification. During the formation of the double extended target primer, with every thermal cycle a significant amount of primer is expected to be removed by primer-primer hybridization and extension. As such, in some embodiments, it can be advantageous to use relatively high levels of primer.

In some embodiments, the target primer comprises, consists, or consists essentially of a relatively short 3′ target specific regions, such as a short 3′ random region. In some embodiments, this shorter 3′ target specific region is used in whole genome amplification where one starts with a low amount of DNA. In some embodiments, the 3′ target specific region is less than 12 nucleotides in length, for example, 11, 10, 9, 8, 7, 6, 5, 4, or 3 nucleotides. In some embodiments, the 3′ target specific region is between 9 and 2, 8 and 3, 7 and 3, 6 and 3, or 6 and 4 nucleotides in length.

In some situations, after incorporation of a universal region, universal primers will still have a problem of having some homology with internal sequences in highly complex populations of long gDNA fragments from the whole genome. Where the concentration of the universal primers are typically on a μM scale, even partial matches of the 3′ end of the universal primers with internal sequences of gDNA fragments can generate shorter products. These shorter products can be preferentially amplified by high concentrations of universal primers. Thus, some of the present embodiments can be used to limit the generation of these short products from primer-dimers or spurious internal priming. In some embodiments long tracts of dT bases can be used in the target primer (as a noncomplementary region for example) for the above reason and because the frequency of poly dT in the middle of gDNAs can be low. In other embodiments, tracts of sequences rarely found in the target genome are used as a noncomplementary region.

As will be appreciate by one of skill in the art, while the 3′ target specific region often includes a random or degenerate region, in some embodiments, the sequence is a specific sequence or collection of specific sequences. In some embodiments, the target primer can include additional sequence sections to those described above. In other embodiments, the target primer only includes those sections depicted in FIG. 1A and/or 1C. Additionally, as will be appreciated by one of skill in the art, some of the presently disclosed techniques can be applied to RNA amplification as well, for example, by including an initial reverse transcription step.

As will be appreciated by one of skill in the art, in some embodiments, a noncomplementary region is used throughout numerous primers, allowing for multiple primers, such as primers including universal, random, or degenerate regions, to be used with a reduced risk of undesired priming events. This can be useful in multiplexed reactions in which numerous different starting primers are employed.

In some embodiments, the above methods can allow for a significant amount of amplification to occur. In some embodiments, the amplification is of nucleic acid sequences of a significant length (e.g., 200 or more nucleic acids). In some embodiments, the amplification of these lengths of target nucleic acid sequences, across a genome's worth of nucleic acid sequence, is achieved. In some embodiments, at least a fraction of the genome is amplified, e.g., 0-1, 1-5, 5-10, 10-20, 20-30, 30-40, 40-50, 50-60, 60-70, 70-80, 80-90, 90-95, 95-99, or 99-100% of the genome is amplified. In some embodiments, at least some fraction of the fraction amplified is of the desired length, e.g., 0-1, 1-5, 5-10, 10-20, 20-30, 30-40, 40-50, 50-60, 60-70, 70-80, 80-90, 90-95, 95-99, or 99-100% is at least 200 bp in length.

In some embodiments, the amount of amplification across a genome is substantially similar. In some embodiments, the amount of amplification for the various target nucleic acids sequences is the same. In other words, sequences A-Z are all amplified to a similar extent so that the resulting ratio of product nucleic acid sequences is the substantially the same for sequences A-Z. In some embodiments, the ratios are maintained in a qualitative manner (e.g., there is more of sequence A than sequence B).

In some embodiments, the amount of amplification of the desired fragments that is achieved is substantial. For example, amplification of the initial product over 30 fold can be achieved, e.g., 30-100, 100-1000, 1,000-3000, 3000-10,000, 10,000-50,000, 50,000-100,000, 100,000-500,000, 500,000-800,000, 800,000-1,000,000, 1,000,000-10,000,000 fold or more. In some embodiments this is achieved with a reduced amount of primer dimer formation and/or spurious priming. In some embodiments, the amount of primer dimers is reduced by at least some amount, e.g., 0-1, 1-5, 5-10, 10-20, 20-30, 30-40, 40-50, 50-60, 60-70, 70-80, 80-90, 90-95, 95-99, or 99-100%.

As noted above, some of the embodiments can be advantageously used when random and/or degenerate priming regions are employed at the 3′ target specific region of the primer, when universal primers are used, or when both aspects are used. Moreover, in some embodiments, further benefits can be obtained when numerous such primers (or other non-target primers) are combined within a reaction (such as in multiplexed or subsequent amplification or extension reactions). As such, as noted above, some of the embodiments can be useful for whole genome amplification. However, not all of the disclosed embodiments are limited to such applications. Even amplification reactions that do not include random regions, or do not involve whole genome amplification can benefit from some of the above embodiments. For example, some of the above embodiments will reduce the number or amount of relatively short nucleic acid sequences that are amplified from a target. As will be appreciated by one of skill in the art, these shorter sequences can be problematic for a variety of reasons (e.g., since they are shorter, they will dominate subsequent amplification reactions). Additionally, the insertion of the noncomplementary region generally allows for one to use either a random, specific, or mix thereof, region for target hybridization, while reducing the likelihood that the target sequence will hybridize too frequently or nonspecifically.

In some embodiments, the target primers and relevant methods are employed in massively multiplexed procedures in which various target primers are employed. As will be appreciated by one of skill in the art, the above embodiments employing degenerate ends at the 3′ target specific region of the probe is one form of multiplexing. However, in some embodiments, different sequences are also employed within the target primer so as to provide a degree of separation or distinctness among the amplified products. In some embodiments, these different sequences are in the universal priming section, a tag sequence, or other additional section added to the target primer. In some embodiments, the number of primers having these different sequences (apart from differences in the 3′ target specific region) are at least 2, if not more, for example, 2-5, 5-10, 10-20, 20-30, 30-50, 50-100, 100-200, or more primers can be used. In some embodiments, the primers can include specific bar-code sequences to allow for ease of identification.

Aspects of the present teachings may be further understood in light of the following examples, which should not be construed as limiting the scope of the present teachings in any way.

### Example 1

### MDA Hybrid Amplification

This example describes how one can employ target primers for the amplification of a substantial portion of a genome.

First, one obtains, provides, or is provided a sample that includes genomic DNA. The genomic DNA in the sample is isolated from various non-DNA impurities in the sample, if necessary.

Following this, a target primer is added to the solution containing the gDNA. The target primer can include a degenerate section and therefore having a plurality of said target primers actually comprises numerous primers, each having a different 3′ target specific sequence. The target primer is then extended via a MDA reaction using phi 29.

Prior to the formation of a significant amount of a hyper-branched product, the MDA reaction is stopped by raising the temperature of the reaction solution to 65° C. A PCR reaction is then performed using a target primer. The target primer for the PCR and MDA reactions can be the same (although the target primers for the MDA reaction can be designed for MDA reactions, e.g., to include at least one or two phosphothioate bonds on the 3′ end).

Thus, a double-extended target primer is eventually formed.

Following this, an amplification process can be performed with one or more amplification primers. Each amplification primer includes a section that is substantially identical in sequence to the universal region in the original target primer.

Following this, a digest is optionally performed on the solution so that any single stranded primers are eliminated. This can be achieved, for example, by the addition of exonuclease I.

Following this, the conditions of the solution are adjusted, if necessary, to allow some of the amplified double-extended target primer to self-hybridize (at least the double-extended target primers that are the primer-dimers).

Insert amplification primers are then added to the solution. The insert amplification primers can be degenerate primers, primers to a desired sequence, and/or universal primers.

The amplified double-extended target primer can also be divided into separate containers (such as wells) and a specific insert amplification primer (or primer set) added to each container to allow amplification to occur based on that specific insert amplification primer (or set). Numerous such insert amplification primers can be used in series or parallel in the separate containers. A PCR amplification is performed on the solution (or more specifically for each solution) under conditions that allow the annealing and extension of the insert amplification primers, while keeping the conditions such that the undesired double-extended target primers are selectively self-hybridized when the annealing step involving the insert primer occurs.

The above steps will result in the amplification of the target nucleic acid sequence.

### Example 2

### MDA Hybrid Amplification

This example describes how one can employ target primers for the amplification of a substantial portion of a genome.

First, one obtains, provides, or is provided a sample that includes genomic DNA. The genomic DNA in the sample is isolated from various non-DNA impurities in the sample, if necessary.

Following this, a target primer that includes a universal region and a 3′ target specific region is added to the solution containing the gDNA. The target primer can include a degenerate section and therefore actually comprise numerous primers, each having a different 3′ target specific sequence. The target primer will include a 3′ end that is resistant to 3′ exonuclease. The target primer is then extended at a constant temperature using a highly processive enzyme.

After a desired period of time (sufficient to produce a desired amplification of the target sequence), the MDA reaction is attenuated. A PCR reaction is then performed using a target primer (which can lack the phosphothioate bonds that are specific to the MDA primers).

Thus, a double-extended target primer is formed.

Following this, the method can proceed as outlined in Example 1.

The above steps will result in the amplification of the target nucleic acid sequence.

### Example 3

### MDA Amplification

This example describes how one can employ target primers for the amplification of a substantial portion of a genome.

First, one obtains, provides, or is provided a sample that includes genomic DNA. The genomic DNA in the sample is isolated from various non-DNA impurities in the sample, if necessary.

Following this, a target primer that includes a universal region and a 3′ target specific region is added to the solution containing the gDNA. The target primer includes a degenerate section and therefore actually comprise numerous primers, each having a different 3′ target specific sequence. The target primer includes a 3′ end that is resistant to 3′ exonuclease. The target primer is then extended at a constant temperature using a highly processive enzyme, via a MDA reaction.

The MDA reaction is allowed to continue until at least some double-extended target primers are formed, each including the MDA primer on one end and a complement of the MDA primer on the other end. Thus, a double-extended target primer is formed.

Following this, an amplification step can be performed with one or more amplification primers. Each amplification primer includes a section that is substantially identical in sequence to the universal region in the original target primer.

Following this, a digest is optionally performed on the solution so that any single stranded primers are eliminated. This can be achieved via treatment with or exposure to exonuclease I.

Following this, the conditions of the solution are adjusted, if necessary, to allow the amplified double-extended target primer to self-hybridize (at least the double-extended target primers that are the primer-dimers).

Insert amplification primers are then added to the solution. The insert amplification primers can be degenerate primers or universal primers.

The amplified double-extended target primer can also be divided into separate containers (such as wells) and a specific insert amplification primer (or primer set) added to each container to allow amplification to occur based on that specific insert amplification primer (or set). Numerous such insert amplification primers can be used in series or parallel in the separate containers. A PCR amplification is performed on the solution (or more specifically for each solution) under conditions that allow the annealing and extension of the insert amplification primers, while keeping the conditions such that the undesired double-extended target primers are selectively self-hybridized.

The above steps will result in the amplification of the target nucleic acid sequence.

### Example 4

### Insert Amplification—Primer Pools

As will be appreciated by one of skill in the art, insert amplification can be achieved based on knowing which sequence was (or should be) contained within the insert, such as RNase P. In situations in which the target within the insert is not initially known, such as when an entire genome is being amplified, the protocol can be varied slightly to take this variable into account. For example, indiscriminant primers could be used. Alternatively, and as described in this example, numerous primers can be tested or used on the amplified sample.

Following any of the above initial amplification procedures (e.g., at a point following the formation of the double-extended target primer, but prior to the use of an insert amplification primer) one can divide the amplified product into numerous subsamples. Each subsample will simply be a fraction of the amplified product, and thus can include a representative (e.g., proportionate and substantially complete) distribution of the various double-extended target primers. Each subsample can be placed in a separate well, to which a specific known, or knowable, insert amplification primer, or primers, can be added. Following this, an amplification step can be performed in each of the wells. This will allow for the amplification of the insert section of the self-hybridized double-extended target primer. These amplified sequences can then be detected, such as by sequencing.

As will be appreciated by one of skill in the art, numerous insert amplification primers can be used for the above processing, e.g., 2-10, 10-50, 50-100, 100-1000, 1000-10,000, 10,000-30,000, 30,000-40,000, 40,000-50,000, 50,000-100,000, or more primers. Each can be used in a separate well with a representative portion of the amplified target nucleic acid sequence. As will be appreciated by one of skill in the art, during the amplification, the conditions should be such that the double-extended target primer is self-hybridized, resulting in the selective amplification of the initially amplified products of the desired size.

### Example 5

### STR Amplification

The present example demonstrates how one can use the methods and primers described herein to amplify a STR locus of interest.

At least one target primer, having a 3′ target specific region that will bind near a locus to be examined, is combined with a sample that includes a target nucleic acid sequence. The 3′ target specific region can be selected so that it binds near at least one of the following loci: TH01, TPOX, CSF1PO, vWA, FGA, D3S1358, D5S818, D7S820, D13S317, D16S539, D8S1179, D18S51, D21S11, D2S1338, D3S1539, D4S2368, D9S930, D10S1239, D14S118, D14S548, D14S562, D16S490, D16S753, D17S1298, D17S1299, D19S253, D19S433, D20S481, D22S683, HUMCSF1PO, HUMTPOX, HUMTH01, HUMF13AO1, HUMBFXIII, HUMLIPOL, HUMvWFA31, Amelogenin, D12s391, D6S1043, SE33, or any combination thereof. The amplification outlined in any of the above examples or embodiments can be performed, thereby resulting in the amplification of the relevant locus.

### Example 6

### STR Amplification

The present example demonstrates how one can use the methods and primers described herein to amplify a STR locus of interest.

At least one target primer having a 3′ target specific region that comprises a degenerate region, is combined with a sample that includes a target nucleic acid sequence. The target primer is used to amplify the target nucleic acid sequence as provided in any of the above examples. However, once the double extended primer is created, the insert amplification primers that are used are selected so that the insert amplification primers bind near at least one of the following loci: TH01, TPOX, CSF1PO, vWA, FGA, D3S1358, D5S818, D7S820, D13S317, D16S539, D8S1179, D18S51, D21S11, D2S1338, D3S1539, D4S2368, D9S930, D10S1239, D14S118, D14S548, D14S562, D16S490, D16S753, D17S1298, D17S1299, D19S253, D19S433, D20S481, D22S683, HUMCSF1PO, HUMTPOX, HUMTH01, HUMF13AO1, HUMBFXIII, HUMLIPOL, HUMvWFA31, Amelogenin, D12s391, D6S1043, and SE33. This will then allow for the amplification of the STR at the relevant locus.

The present disclosure clearly establishes that the presently disclosed processes can be effective in selectively amplifying usefully sized fragments throughout relatively long stretches of gDNA from a target sample. While any of the above embodiments may have been described in terms of a linear primer, in other embodiments, the initial primer can be looped or need not be linear (as long as there is a universal region that is placed on one end and its complement is placed on the other end of a section of nucleic acid to be amplified. Thus, in some embodiments, any or every one of the above embodiments can be used with a stem-looped primer (or “loopable” primer) instead of a linear primer.

Furthermore, in some embodiments, the amount of amplification is, compared to the current state of the art, very high (approximately 3000 fold to over hundreds of thousands fold), while still amplifying the larger fragments. This is in contrast to previous attempts at amplification using random primers that appeared to generally reach lower levels of amplification. (See, e.g., Zhang et al., PNAS, vol. 89, 5847-5851, (1992), approximately 30 fold; and Genomeplex® Whole Genome Amplification (WGA) Kit by Sigma-Aldrich, discussed on the world wide web at biocompare.com/review/769/Genomeplex-Whole-Genome-Amplification-(WGA)-Kit-by-Sigma-Aldrich.html, discussing 3000 fold). Additionally, as shown above, the amplification ability can be enhanced through the use of an Exo I digestion step, although this is clearly not required. It is believed that these data demonstrate that 3′ target-specific portions (e.g., degenerate regions) of 7-15 nucleic acids in length will work for some embodiments. Additionally, in some embodiments these relatively large increases in amplification are achieved while still maintaining some degree of dose response during the amplification. For example, in some embodiments, relatively small amounts of one species to be amplified will still be a relatively small percent of the amplified product (although it could have been amplified, e.g., 100-1,000,000 times).

In this disclosure, the use of the singular can include the plural unless specifically stated otherwise or unless, as will be understood by one of skill in the art in light of the present disclosure, the singular is the only functional embodiment. Thus, for example, “a” can mean more than one, and “one embodiment” can mean that the description applies to multiple embodiments. The phrase “and/or” denotes a shorthand way of indicating that the specific combination is contemplated in combination and, separately, in the alternative.

The section headings used herein are for organizational purposes only and are not to be construed as limiting the described subject matter in any way.

It will be appreciated that there is an implied “about” prior to the temperatures, concentrations, times, etc. discussed in the present teachings, such that slight and insubstantial deviations are within the scope of the present teachings herein. For example, “a primer” means that more than one primer can, but need not, be present; for example but without limitation, one or more copies of a particular primer species, as well as one or more versions of a particular primer type, for example but not limited to, a multiplicity of different target primers. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the invention.

Unless specifically noted in the above specification, embodiments in the above specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims).

### Incorporation by Reference

All references cited herein, including patents, patent applications, papers, text books, and the like, and the references cited therein, to the extent that they are not already, are hereby incorporated by reference in their entirety. In the event that one or more of the incorporated literature and similar materials differs from or contradicts this application; including but not limited to defined terms, term usage, described techniques, or the like, this application controls.

### EQUIVALENTS

The foregoing description and Examples detail certain preferred embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

What is claimed is:

1. A method for targeted genome wide nucleic acid sequence amplification, said method comprising the following processes:
(a) providing at least one first target primer, wherein said first target primer comprises a 3′ target specific region and a universal region;
(b) contacting said first target primer and a target nucleic acid sequence such that said 3′ target specific region hybridizes to said first target nucleic acid sequence;
(c) performing isothermal multiple strand displacement amplification (MDA) of said target nucleic acid sequence using said first target primer; and
(d) forming a double-extended primer comprising said universal region on one end and a sequence that is complementary to said universal region on the opposite end, and further comprising an insert section in between said ends.

2. The method of claim 1, further comprising the processes of adding at least a second primer that is complementary to a sequence within said insert section; and performing PCR amplification of said insert section.

3. The method of claim 1, wherein said MDA is performed using phi 29 polymerase.

4. The method of claim 1, wherein said forming a double-extended primer involves PCR amplification.

5. The method of claim 2, wherein said PCR amplification is performed using Taq polymerase.

6. The method of claim 2, wherein only a single PCR primer sequence is employed to amplify any and/or all PCR amplified nucleic acid sequences.

7. The method of claim 2, wherein said PCR amplification occurs immediately after said MDA process.

8. The method of claim 2, further comprising a process of terminating said MDA process by an increase in temperature.

9. The method of claim 8, wherein said increase in temperature is part of said PCR amplification process.

10. The method of claim 1, wherein said target primer is a loopable primer.

11. The method of claim 1, wherein said target primer is a linear primer.

12. The method of claim 1, whereby one reduces primer-related background amplification resulting from said MDA process, while retaining relatively even gene amplification during said MDA process.

13. The method of claim 1, wherein said target nucleic acid sequence is derived from a whole genome.

14. The method of claim 1, wherein said universal region does not hybridize to said target nucleic acid sequence.

15. The method of claim 1, wherein said 3′ target specific region comprises a degenerate region.

16. The method of claim 1, wherein said target primer is a linear primer when it hybridizes to said target nucleic acid sequence.

17. The method of claim 1, wherein said target primer is a looped primer when it hybridizes to said target nucleic acid sequence.

18. The method of claim 1, wherein said processes (a)-(d) occur in the order in which they are listed.

19. The method of claim 1, wherein said double-extended primer is formed during process (c).

20. The method of claim 1, wherein said process (c) occurs in the presence of a PCR amplification enzyme.

21. The method of claim 1, further comprising a process of allowing said double-extended primer to self-hybridize via hybridization of said universal region to said sequence that is complementary to said universal region.

22. The method of claim 1, further comprising a process of adding a third primer that is complementary to a sequence within said insert section.

23. The method of claim 1, wherein said providing is of at least two first target primers, wherein each of said first target primers has the same universal region, and wherein each of said first target primers has a different sequence at their 3′ target specific region.

24. The method of claim 23, wherein multiple copies of each of said first target primers are employed.

25. The method of claim 2, further comprising a process of adding additional first target primer comprising a universal region and a 3′ target specific region, wherein said 3′ target specific region comprises a degenerate region and wherein said additional first target primer is added after said isothermal MDA process and before said PCR process.

26. The method of claim 1, wherein said first target primer comprises at least one phosphothioate bond at its 3′ end.

27. The method of claim 1, wherein said first target primer comprises at least two phosphothioate bonds at its 3′ end.

28. The method of claim 1, further comprising a process of performing PCR amplification using a second target primer.

29. The method of claim 1, further comprising a process of performing amplification using an amplification primer that comprises a universal region.

30. The method of claim 1, further comprising a process of performing PCR amplification using a second target primer prior to formation of a hyper-branched product during said isothermal MDA process.

31. The method of claim 1, wherein said target nucleic acid sequence is produced from a reverse transcription reaction.

32. A method for genome wide nucleic acid sequence amplification, said method comprising the following steps:
(a) providing a target primer, wherein said target primer comprises a 3′ target specific region and a universal region, wherein the 3′ target specific region comprises a degenerate sequence;
(b) contacting said target primer to a target nucleic acid sequence such that said 3′ target specific region hybridizes to said target nucleic acid sequence;
(c) performing an isothermal multiple strand displacement amplification (MDA) on said target nucleic acid sequence using said target primer;
(d) performing a PCR amplification, wherein said PCR amplification is after said isothermal MDA, but prior to formation of a significant amount of a hyper-branched product of said isothermal MDA, thereby forming a double-extended target primer comprising said universal region on one end and a sequence that is complementary to said universal region on the opposite end;
(e) performing an amplification of said double extended target primer using an amplification primer, said amplification primer comprising a universal region;
(f) adding at least a first and second insert primer; and
(g) performing PCR amplification within an insert section, using said first and second insert primers.

33. A PCR primer kit, said kit comprising:
a target primer comprising:
a universal region, wherein said universal region comprises a nucleic acid sequence that has an appropriate Tm to serve as a primer, that has an appropriate GC content to serve as a primer, and wherein said universal region comprises 12 to 35 bases; and
a 3′ target specific region located in the 3′ direction from said universal region, wherein said 3′ target specific region comprises at least 2 bonds that are phosphothioate bonds.

34. The PCR primer kit of claim 36, further comprising an amplification primer comprising said universal region, wherein said amplification primer lacks the 3′ target specific region.

35. The PCR primer kit of claim 37, further comprising an isothermal MDA enzyme.

36. The PCR primer kit of claim 38, further comprising a PCR enzyme.

37. The PCR primer kit of claim 39, further comprising at least one insert amplification primer.
